(19) United States
(12) Patent Application Publication
(10) Pub. No.: US 2002/0150968 A1
(43) Pub. Date: Oct. 17, 2002

(54) Glycoconjugate and sugar nucleotide synthesis using solid supports

(76) Inventors: Peng G. Wang, Troy, MI (US); Xi Chen, Norristown, PA (US)

Correspondence Address: Brinks Hofer Gilson & Lione, P.O. Box 10395, Chicago, IL, 60610, US

(21) Appl. No.: 09/757,846

(22) Filed: Jan. 10, 2001

(51) Int. Cl. C12P 33/20; C12P 21/02; C12P 19/20; C12N 11/18

(52) U.S. Cl. 435/53; 435/68.1; 435/96; 435/175

(57) ABSTRACT

This invention relates to methods and compositions for the in vitro production of glycoconjugates. In particular, a preferred production system is provided that comprises a solid support, at least one sugar nucleotide producing enzyme, at least one glycosyltransferase, at least one bioenergetic, and at least one acceptor. The sugar nucleotide producing enzyme(s) is preferably immobilized on the solid support. The glycosyltransferase may be co-immobilized on the solid support with the sugar nucleotide producing enzyme(s), or may be provided to the solid support in solution.

### FEDERALLY SPONSORED RESEARCH OR DEVELOPMENT

The U.S. Government may have rights in the present invention pursuant to the terms of grant number A1 44040 awarded by the National Institutes of Health.

### BACKGROUND OF THE INVENTION

Advances in biological science have demonstrated that carbohydrates serve not only as energy sources or structural components, but also as key elements in a variety of molecular recognition, communication, and signal transduction events. Functions include attachment points for antibodies (e.g., human blood type A and B antigens and α-Gal glycoconjugates), receptor sites for bacterial and viral infections, cell adhesion sites for inflammation (e.g., sialyl-Lewis X antigen), and involvement in metastasis. Additionally, carbohydrates play a role in cell differentiation, development, regulation (e.g., gangliosides), protein folding (e.g., N-linked and O-linked glycan), and non-immunological defense (e.g., human milk glycoconjugate).

Despite the important biological functions and increasing demand for glycoconjugates, both chemical and enzymatic syntheses of glycoconjugates have been difficult. Large-scale production of glycoconjugates by chemical methods requires tedious protection and deprotection steps. Also, due to the high cost of necessary sugar nucleotides, glycoconjugates longer than a trisaccharide are not economically feasible using traditional chemical methods. Enzymatic synthesis of glycoconjugates using glycosidase-catalyzed transglycosylation reactions suffers from low yields and unpredictable regio-selectivity.

Glycosyltransferases from the Leloir pathway, which are highly specific in the formation of glycosides, have proven to be a viable strategic choice for the preparative synthesis of oligosaccharides. Although a vast number of glycosyltransferases have been cloned from eukaryotic and bacterial sources, the limited access to recombinant glycosyltransferases and the prohibitive cost of the sugar-nucleotide donors prevent their application in large-scale synthesis. Thus, there remains a need for large-scale, industrial production of oligosaccharides, and glycoconjugates in general.

In 1998, Kyowa Hakko Inc. in Japan made a significant breakthrough in large-scale synthesis of carbohydrates (Koizumi, S. et al., Nature Biotech. 1998, 16: 847-850). The key in Kyowa Hakko's technology for the large-scale production of UDP-galactose and Galα1,4Lac globotriose was a C. ammoniagenes bacterial strain engineered to efficiently convert inexpensive orotic acid to UTP. When combined with an E. coli strain engineered to over-express UDP-galactose biosynthetic genes including galK (galactokinase), galT (galactose-1-phosphate uridyltransferase), galU (glucose-1-phosphate uridyltransferase), and ppa (pyrophosphatase), UDP-galactose accumulated in the reaction solution. Combining these two strains with another recombinant E. coli strain over-expressing α1,4-galactosyltransferase gene of Neisseria gonorrhoeae, high concentration of globotriose was obtained.

The same UDP-galactose production system was also successfully applied in the large-scale production of disaccharide LacNAc (Endo, T. et al., Carbohydr. Res. 1999, 316: 179-183). UDP-N-acetylglucosamine (UDP-GlcNAc) and CMP-sialic acid have been produced through a similar methodology (Tabata, K. et al., Biotech, Lett. 2000, 22: 479-483; Endo, T. et al., Appl. Microbiol. Biotechnol. 2000, 53: 257-261). The Kyowa Hakko technology is also described in EP 0861902 and EP 0870841.

Recent advances have demonstrated that production of glycoconjugates in a cell-free environment is possible. (e.g., Fujita, K. et al. Biochem. Biophys. Res. Commun. 2000, 267:134; Revers, L. et al., Biochim. Biophys. Acta 1999, 1428: 88; Lubineau, A. et al., Carbohydr. Res. 1997, 300: 161; Asano, N. et al., Carbohydr. Res. 1994, 258: 255; Roth, S. U.S. Pat. No. 5,583,042, 1996; Roth, S. U.S. Pat. No. 5,288,637, 1994; Roth, S. U.S. Pat. No. 5,180,674, 1993; Thiem, J. and Wiemann, T. Synthesis January/February 1992: 141-145.) Wong and Whitesides utilized a two-step process to create solid supports having enzymes for glycoconjugate synthesis deposited thereon. (Wong, C.-H., et al. J. Org. Chem. 1982, 47: 5416-5418.)

Despite the significant breakthroughs of Kyowa Hakko and Wong and Whitesides, drawbacks remain. The Kyowa Hakko processes require: (1) the use of recombinant bacteria; (2) several plasmids in several bacterial strains; (2) transportation of intermediates in and out of the bacterial membrane to be utilized by the next enzyme; and (3) nucleotide derivatives. The in vitro system of Wong and Whitesides requires a two-step procedure for creating the solid support. The enzymes must be purified, and subsequently immobilized onto the solid support. Furthermore, the solid supports of the Wong and Whitesides system can only be used in a small number of production cycles, possibly as few as two or three, before they lose activity. Considering these drawbacks of existing technologies, there remains a need for simpler, cost-effective processes of producing oligosaccharides and other glycoconjugates.

### BRIEF SUMMARY OF THE INVENTION

The present invention overcomes the deficiencies of the prior art. Provided are processes and compositions for the inexpensive, large-scale synthesis of glycoconjugates. The present invention provides one or more of the following advantages (1) inexpensive, large-scale synthesis of glycoconjugates; (2) one-step purification and immobilization of necessary enzymes on a solid support; (3) increased enzyme stability; (4) easy separation of product and enzymes from a reaction solution; (5) cell-free system that eliminates problems associated with transporting large glycoconjugates across bacterial membranes; (6) regeneration of sugar nucleotides; (7) pre-purification of enzymes is not necessary; (8) the enzymes can be recycled for multiple uses; (9) the solid support can be recycled for multiple uses, and (10) the versatility of the system allows easy combination between different sugar nucleotide regeneration systems and different glycosyltransferases, allowing production of a wide spectrum of glycoconjugates.

Described herein are:

1.) Methods of producing oligosaccharides and other glycoconjugates;

2.) Microspherical beads with immobilized sugar-nucleotide production and/or regeneration enzymes, either alone or coimmobilized with one or more glycosyltransferase enzymes; Systems for producing glycoconjugates; and

3.) Kits containing various types of beads and/or other material for use in the production of oligosaccharides or other glycoconjugates in accordance with the present invention.

In one aspect, the present invention embodies an in-vitro glycoconjugate-producing system, comprising a solid support, one or more sugar nucleotide producing or regenerating enzymes immobilized on the solid support, and one or more glycosyltransferase enzymes immobilized on the solid support. In another aspect, the invention embodies a reaction vessel comprising a solid support, one or more sugar nucleotide producing or regenerating enzymes immobilized on the solid support, and one or more glycosyltransferase. The glycosyltransferase may be immobilized on the solid support, or may be in solution. In another aspect, the invention provides a method of producing a glycoconjugate, comprising the step of contacting a reaction vessel containing a solid support, one or more sugar nucleotide producing or regenerating enzymes, and one or more glycosyltransferase with a bioenergetic, an acceptor, and a precursor. In another aspect, the present invention provides kits comprising a solid support, one or more sugar nucleotide producing or regenerating enzymes, and one or more glycosyltransferase. In the kit, one or more of the enzymes is immobilized on the solid support. The kit can also contain additional items, such as plasmids and cells. Also, in one aspect, the invention embodies a population of beads having at least one bead with one or more sugar nucleotide producing or regenerating enzymes immobilized on the bead, and at least one bead having one or more glycosyltransferase immobilized on the bead.

### BRIEF DESCRIPTION OF SEVERAL VIEWS OF THE DRAWINGS

FIG. 1 Metabolic biopathway for the synthesis of α-Gal. Five enzymes are involved including α1,3GalT (α1,3-galactosyltransferase, EC 2.4.1.151), GalK (galactokinase, EC 2.7.1.6), GalT (galactose-1-phosphate uridylyltransferase, EC 2.7.7.10), GalU (glucose-1-phosphate uridylyltransferase, EC 2.7.7.9), and PykF (pyruvate kinase, EC 2.7.1.40). Metal cofactors required by individual enzymes are shown.

FIG. 2 Plasmid map of an α-Gal engineered vector harboring five genes encoding enzymes involved in the biosynthetic pathway of UDP-Gal regeneration and the production of α-Gal oligosaccharides. Introduced restriction enzyme sites: EcoR I, Sac II, Sal I, Xba I, Cla I. Abbreviation: rbs, ribosomal binding site.

FIG. 3 Biosynthetic pathway and corresponding plasmid map for using ATP as a bioenergetic.

FIG. 4 Biosynthetic pathway and corresponding plasmid map for using polyphosphate as a bioenergetic.

FIG. 5 Biosynthetic pathway and corresponding plasmid map for using pyruvate and O₂ as a bioenergetic.

FIG. 6 Biosynthetic pathway and corresponding plasmid for synthesis of glycoconjugates with UDP-Glc regeneration.

FIG. 7 Biosynthetic pathway and corresponding plasmid for synthesis of glycoconjugates with UDP-GlcNAc regeneration.

FIG. 8 Biosynthetic pathway and corresponding plasmid for synthesis of glycoconjugates with UDP-GalNAc regeneration.

FIG. 9 Biosynthetic pathway and corresponding plasmid for synthesis of glycoconjugates with UDP-GlcA regeneration.

FIG. 10 Biosynthetic pathway and corresponding plasmid for synthesis of glycoconjugates with CMP-NeuNAc regeneration.

FIG. 11 Biosynthetic pathway and corresponding plasmid for synthesis of glycoconjugates with GDP-Man regeneration.

FIG. 12 Biosynthetic pathway and corresponding plasmid for synthesis of glycoconjugates with GDP-Fuc regeneration.

FIG. 13 Plasmids for the regeneration of UDP-GlcNAc and UDP-GlcA that, when cotransfected into E. coli, are useful to produce hyaluronic acid.

FIG. 14 Exemplary sialic acid containing glycoconjugates.

FIG. 15 Biosynthetic pathway and corresponding plasmid map for synthesis of α-Gal using sucrose as a bioenergetic.

FIG. 16 Helicobacter pylori GDP-fucose-related gene cluster.

FIG. 17 Plasmid for GDP-fucose regeneration.

FIG. 18 Biosynthetic pathway and corresponding plasmid map for synthesis of glucose-terminated glycoconjugate using sucrose synthase.

FIG. 19 Biosynthetic pathway and corresponding plasmid map for synthesis of glucuronic acid-terminated glycoconjugate using sucrose synthase.

FIG. 20 Plasmids for the synthesis of hyaluronon through the regeneration of UDP-GlcNAc and UDP-GlcA using sucrose synthase.

FIG. 21 Biosynthetic pathway and corresponding plasmid for synthesis of glycoconjugates terminated with Galαl,4Gal sequence with UDP-Gal regeneration.

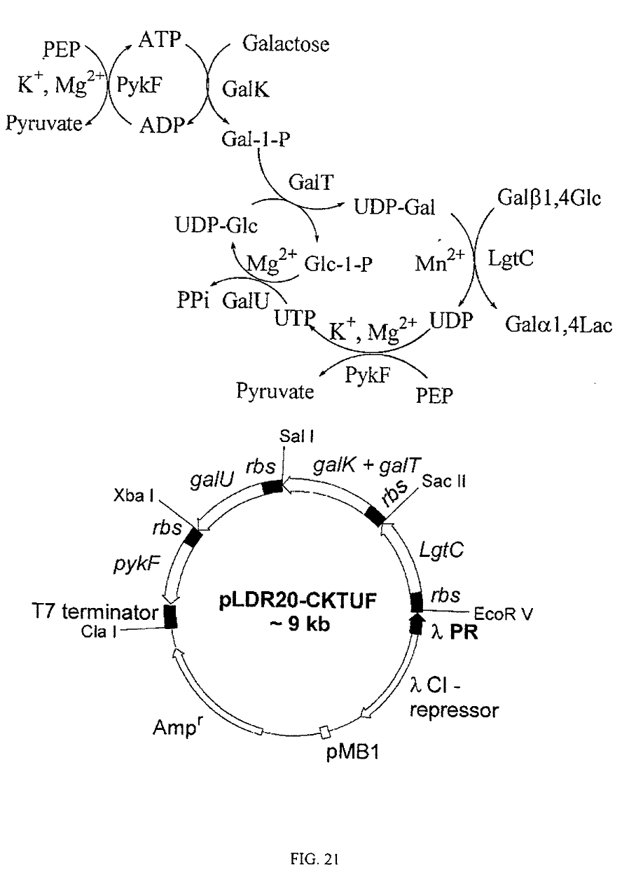

FIG. 22 Biosynthetic pathway and corresponding plasmid map for synthesis of globotriose using sucrose.

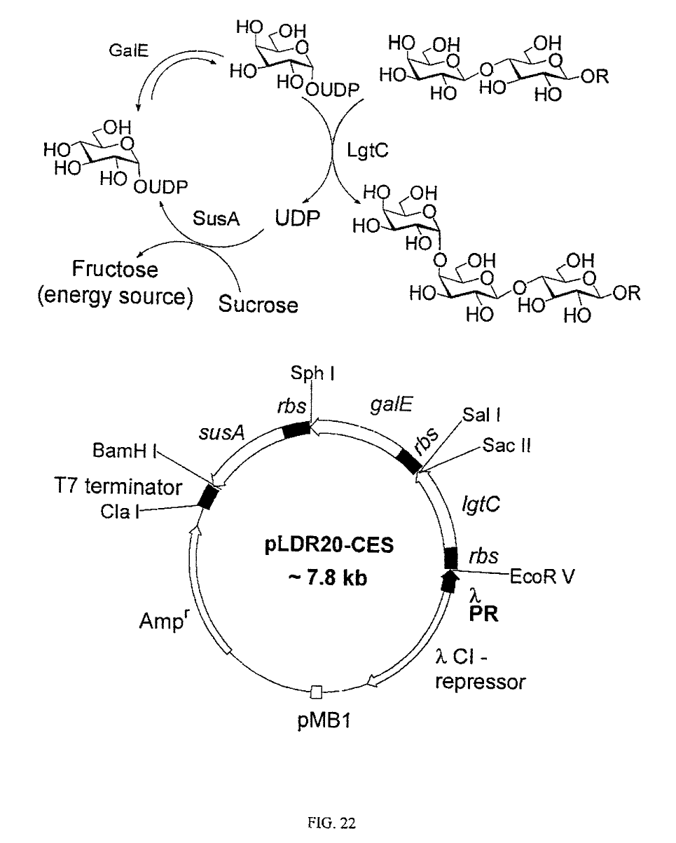

### DETAILED DESCRIPTION OF THE INVENTION

Abbreviations used herein are shown in Table 3 at the end of this disclosure.

The present invention provides a method of producing various glycoconjugates and materials useful in such method. The method utilizes novel compositions and processes. The invention provides microspherical beads that have been engineered to include, by way of immobilization on the bead, enzyme(s) useful in the production of various glycoconjugates. In a preferred embodiment, a bioenergetic, precursor, and acceptor are provided to a bead or a population of beads comprising at least one sugar-nucleotide-regenerating enzyme. The bead may also comprise at least one glycosyltransferase enzyme. If no such glycosyltransferase enzyme is immobilized on the bead, a glycosyltransferase is provided to the bead, either in the reaction solution in which the bead is placed or immobilized on a separate bead. The enzymes on the bead or the population of beads are able to utilize the bioenergetic to produce and/or preferably regenerate a sugar nucleotide. The sugar nucleotide produced and/or regenerated is then utilized by the glycosyltransferase to add the sugar residue to an acceptor, thus producing a glycoconjugate. The acceptor can be any molecular species capable of binding the sugar residue via action by the glycosyltransferase, such as various sugars, proteins, lipids, etc. Consequently, the product of the synthesis can be any appropriate glycoconjugate, such as oligosaccharides, glycoproteins, and glycolipids. In preferred embodiments, an oligosaccharide is produced by the transfer of a sugar residue from a sugar nucleotide to a saccharide acceptor, and the sugar nucleotide is regenerated by the sugar nucleotide regenerating enzyme from the nucleotide resulting from the glycosyltransferase reaction.

### Bioenergetics

An important aspect of the present invention is the ability to produce glycoconjugates in an environment devoid of whole cells. Because biosynthesis of glycoconjugates requires energy, an energy source (bioenergetic) must be provided to the beads of the present invention. Preferably, a sugar nucleotide is directly provided to the beads. Alternatively, however, essentially any bioenergetic that may be converted by enzymes either on the bead or in the reaction mixture to produce a sugar nucleotide may be used. Furthermore, combinations of bioenergetics may be used.

One source of energy that can be utilized is saccharides. Examples of saccharides that may be used as bioenergetics in the present invention include monosaccharides, such as glucose, galactose, fructose, mannose, glyceril, fucose; dissacharides, such as lactose or sucrose; or polysaccharides, such as starch. The saccharides are broken down to produce high-energy phosphate donors, such as ATP, PEP, UTP, GTP, and CTP. The minimum added as an energy source is ATP or PEP by itself. The resulting high-energy phosphate donors may be used by enzymes to produce a sugar nucleotide.

Alternatively, the high-energy phosphate donor itself is provided directly to a bead of the present invention. Because the amount of high-energy phosphate donor produced by providing a saccharide to the bead is limited, directly providing a high-energy phosphate donor to the bead is preferred for large-scale production of glycoconjugates. In preferred embodiments, the high-energy phosphate donor is PEP or ATP. Examples of other preferred bioenergetics include polyphosphate, acetyl phosphate, and sucrose through the function of sucrose synthase.

In preferred embodiments, enzymes beneficial to the utilization of a given bioenergetic are provided to the bead. For example, where PEP is the bioenergetic, pyruvate kinase may be provided to the bead. Pyruvate kinase uses PEP to convert nucleotide diphosphates (UDP, ADP) to nucleotide triphosphates (UTP, ATP). Examples of pyruvate kinases include PykF, PykA, yeast pyruvate kinase (e.g. Burke et al., J Biol Chem 1983, 258(4):2193-201), a rat pyruvate kinase (e.g. Yamada et al., J Biol Chem 1990, 265(32):19885-91), and human pyruvate kinases (e.g. Zarza et al., Haematologica 2000, 85(3):227-32.; Takenaka et al., Eur J Biochem 1991, 198(l):101-6.; Harkins et al., Biochemistry 1977, 16(17):3831-7)

Where ATP is the bioenergetic, nucleotide diphosphate kinase may be provided to the bead. Nucleotide diphosphate kinases, including NdK, from a variety of prokaryotic and eukaryotic sources are known in the art (e.g. Hama, H. et al., Gene 1991, 105: 31-36; Baker and Parker, FEMS Microbiol. Lett. 1994, 121: 293-296; Sundin et al., Mol Microbiol 1996, 20(5):965-79; Ulloa et al., Mol Biochem Parasitol 1995, 70(1-2):119-29; Shimada et al., J Biol Chem 1993, 268(4):2583-9; Ishikawa et al., J Biol Chem 1992, 267(20): 14366-72).

Where polyphosphate is the bioenergetic, polyphosphate kinase may be provided to the bead. Polyphosphate kinases, including PpK, from a variety of prokaryotic and eukaryotic sources are known in the art (e.g. Shiba, T. et al., Biochemistry (Mosc) 2000, 65: 315-323; Van Dien and Keasling, Biotechnol. Prog. 1999, 15: 587-593; Noguchi and Shiba, Biosci. Biotechnol. Biochem. 1998, 62: 1594-1596; Trelstad et al., Appl Environ Microbiol 1999, 65(9):3780-6; Zago et al., Appl Environ. Microbiol. 1999, 65(5):2065-71; Tinsley et al., Infect Immun 1993, 61(9):3703-10; Robinson et al., Biochem Int 1984, 8(6):757-69; J Gen Microbiol 1975, 88(1):65-74).

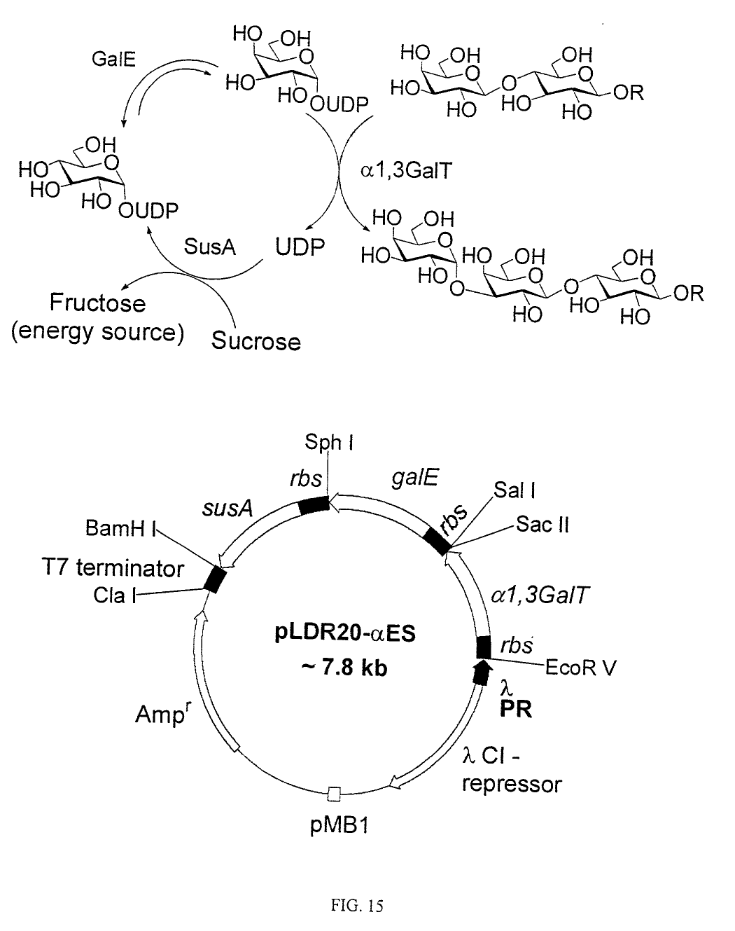

In certain embodiments, sucrose is used as the bioenergetic. Sucrose is a disaccharide consisting of fructose and glucose. Sucrose synthase (UDP-glucose: D-fructose 2-α-D-glucosyltransferase) catalyzes the synthesis and cleavage of sucrose. In some embodiments for the production of α-Gal, the regeneration of UDP-Gal utilizes only two enzymes, sucrose synthase (SS, EC 2.4.1.13) and UDP-Gal 4-Epimerase (GalE, EC 5.1.3.2). Using this UDP-Gal regeneration pathway, the α-Gal synthetic pathway may consist only of three enzymes (FIG. 15). The sucrose synthase is widespread in plant and has been well characterized. Unlike most enzymes of sugar-nucleotide metabolism, SS shows a wide specificity for the nucleoside base.

Sucrose synthase purified from rice grains, together with GalE and β1,4-galactosyltransferae, has been applied in the preparative synthesis of N-acetyllactosamine (LacNAc). A yield of 100% for 10 mM acceptor substrate was obtained under optimized conditions using a repetitive batch technique (Zervosen and Elling, J. Am. Chem. Soc. 1996, 118: 1836-1840). Combined with chemical methods, UDP-N-acetyl-α-D-galactosamine has been obtained using purified sucrose synthase (Bulter et al., Carbohydr. Res. 1997, 305: 469-473). Plant recombinant SS has been obtained and applied in the gram-scale synthesis of ADP-glucose (Zervosen et al., J. Mol. Catalysis B: Enzymatic 1998, 5: 25-28).

The presence of SS has also been demonstrated in several species of green algae (e.g., Duran and Pontis, Mol. Cell Biochem. 1977, 16: 149-152; Salerno, Plant Sci. 1985, 42: 5-8; Salerno, Physiol. Plant 1985, 64: 259-264; Salerno et al., In: Pontis H. G.; Salerno, G. L.; Echeverria, E. J. (eds) Sucrose metabolism biochemistry, physiology and molecular biology, vol 14 (Current Topics in Plant Physiology: An American Society of Plant Physiologist Series), 1995, pp 34-39) and in extracts of Anabaena variabilis, a filamentous heterocystous cyanobacterium (e.g., Schilling and Ehrnsperger, Z. Naturforsch 1985, 40: 776-779). Also, two prokaryotic SS forms (SS-I and SS-II) were purified from Anabaena sp. strain PCC 7119. SS-II was biochemically characterized (Porchia et al., Planta 1999, 210: 34-40) and its gene sequence was reported to GenBank (Acc. # AJ010639). Anabaena SS II was shown to be a tetramer with each subunit having a molecular weight of 92-kDa. Sucrose synthase II exhibited optimal maximum activities between pH 7.5 and 8.2 in the sucrose-synthesis direction, and between 5.9 and 6.5 in the sucrose-cleavage direction. In the sucrose-synthesis direction, either Mg²⁺ or Mn²⁺ increased enzyme activity between 2- and 4-fold using UDP-Glc as substrates. However, the addition of Mn²⁺ strongly inhibited enzyme activity in the sucrose-cleavage direction, while Mg²⁺ has little effect. In the presence of uridine substrate (UDP-Glc or UDP), addition of ATP produced a strong inhibition in both directions.

Where O₂ is the bioenergetic, a series of enzymes for the utilization of O₂ to convert a nucleotide diphosphate to a nucleotide triphosphate may be used. For example, acetate kinase, inorganic pyrophosphatase, and pyruvate oxidase may be used together (Kim and Swartz, Biotech. Bioeng. 1999, 66: 180-188; Grabau, C. et al., J. Biol. Chem. 1989, 264: 12510-12519; Chang and Cronan, Biochemistry 1997, 36: 11564-11573; Wang, A. Y. et al., J. Biol. Chem. 1991, 266: 10959-10966.). Acetate kinases, including AcK (EC 2.7.2.1), from a variety of sources are known in the art (Alm et al., Nature 1999, 397(6715): 176-180; Kahane et al., J Bacteriol. 1979, 137(2): 764-72; Latimer and Ferry, J Bacteriol. 1993, 175(21):6822-9).

Inorganic pyrophosphatases, including PPase (EC 3.6.1.1), from a number of sources may be used in embodiments of the present invention, including those of Heliobacter pylori (Oliva et al., Arch Microbiol 2000, 174(1-2):104-110), Methanococcus jannaschii (Kuhn et al., Arch Biochem Biophys 2000, 379(2):292-8), Bacillus subtilis (Shintani et al., FEBS Lett 1998, 439(3):263-6; Young et al., Microbiology 1998, 144 (Pt 9):2563-71), human (Fairchild et al., Biochim Biophys Acta 1999, 1447(2-3):133-6; Baykov et al., Prog Mol Subcell Biol 1999, 23:127-50), yeast (Pohjanjoki et al., Biochemistry 1998, 37(7):1754-61; Kolakowski et al., Nucleic Acids Res 1988, 16(22):10441-52; Heikinheimo et al., Eur J Biochem 1996, 239(l):138-43), bovine (Yang and Wensel, J Biol Chem 1992, 267(34):24641-7), and plant (Maeshima, Biochim Biophys Acta 2000, 1465(1-2):37-51; Rodrigues, Mol Cell Biol 1999, 19(11):7712-23; Suzuki et al., Plant Cell Physiol 1999, 40(8):900-4).

Pyruvate oxidases, including PoxB (EC 1.2.3.3), from sources such as Lactobacillus plantarum and Pediococcus sp. may be used in certain embodiments of the present invention.

### Sugar-Nucleotide Producing Enzymes

The bioenergetic is utilized by the sugar nucleotide producing enzyme(s) to produce a sugar nucleotide, which is a necessary component for the glycosylation reaction. This sugar nucleotide is used by a glycosyltransferase to add the sugar moiety to an acceptor, such as a saccharide. In preferred embodiments, a precursor is provided to the enzyme(s). The enzyme(s) recognizes the precursor and attaches it to a nucleotide to create the sugar nucleotide (which will act as the donor of the sugar moiety). Of course, in certain embodiments, a non-nucleotide donor molecule may be provided to the enzyme(s) for use by the glycosyltransferase (Lougheed et al., J Biol Chem 1999, 274(53):37717-22.). When the donor is a sugar nucleotide, the end products of the glycosyltransferase reaction are the glycoconjugate with a newly added sugar moiety and either a nucleotide diphosphate or a nucleotide monophosphate. In preferred embodiments, as listed below, some or all of the enzymes necessary for the efficient regeneration of the desired sugar-nucleotide are immobilized on the bead or population of beads. This allows for the continuous production of more sugar-nucleotide for the glycosyltransferase reaction.

An important aspect of the present invention is the ability to tailor the compositions and methods to specific sugar nucleotides. Genes and their respective enzymes involved in sugar-nucleotide generation and regeneration are known in the art (EP 0870841, incorporated herein by reference in its entirety). In light of the present disclosure, one of ordinary skill in the art would understand how to utilize these genes and their respective enzymes to customize the compositions and methods of the present invention to a given sugar nucleotide as discussed below.

Examples of sugar nucleotides that may be regenerated include UDP-Gal, UDP-Glc, UDP-GlcNAc, UDP-GalNAc, UDP-GlcA, CMP-NeuNAc, GDP-Man, GDP-Fuc, and UDP-GalA.

For each of the enzymes, suitable concentrations of substrate can be determined for various quantities of enzyme based upon a variety of parameters, including reaction product.

### A. UDP-Gal

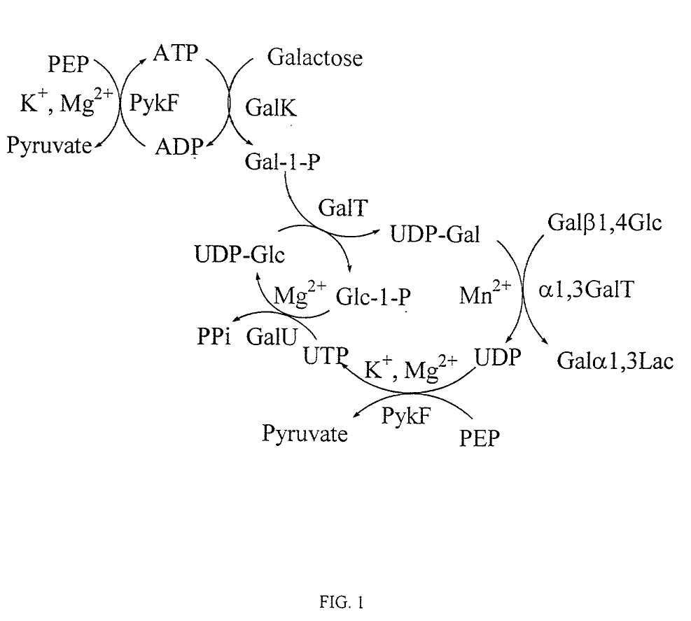

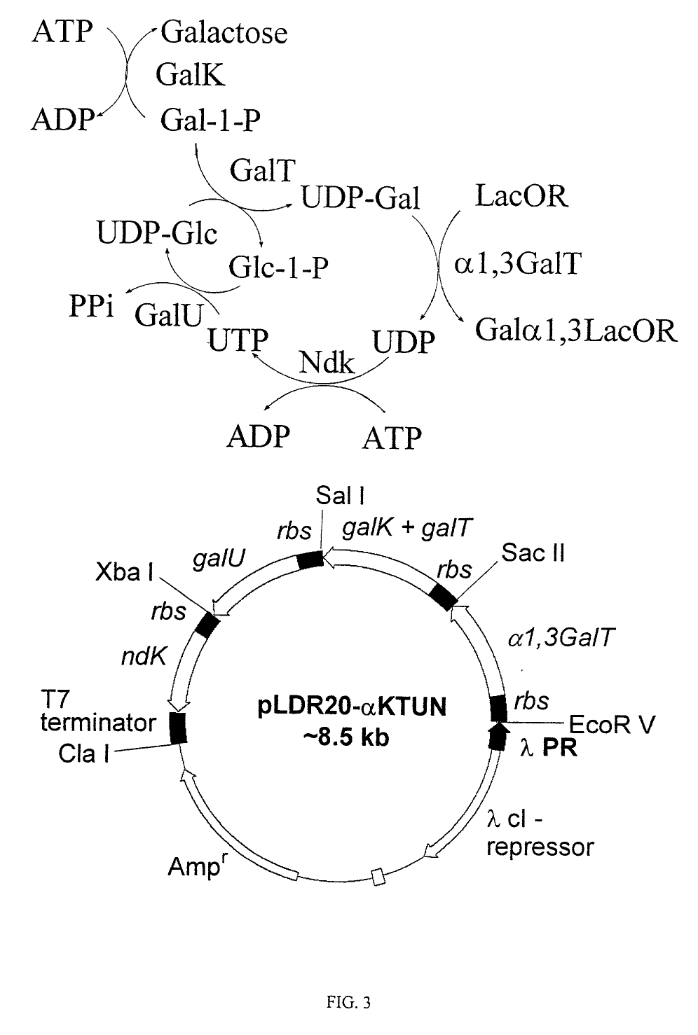

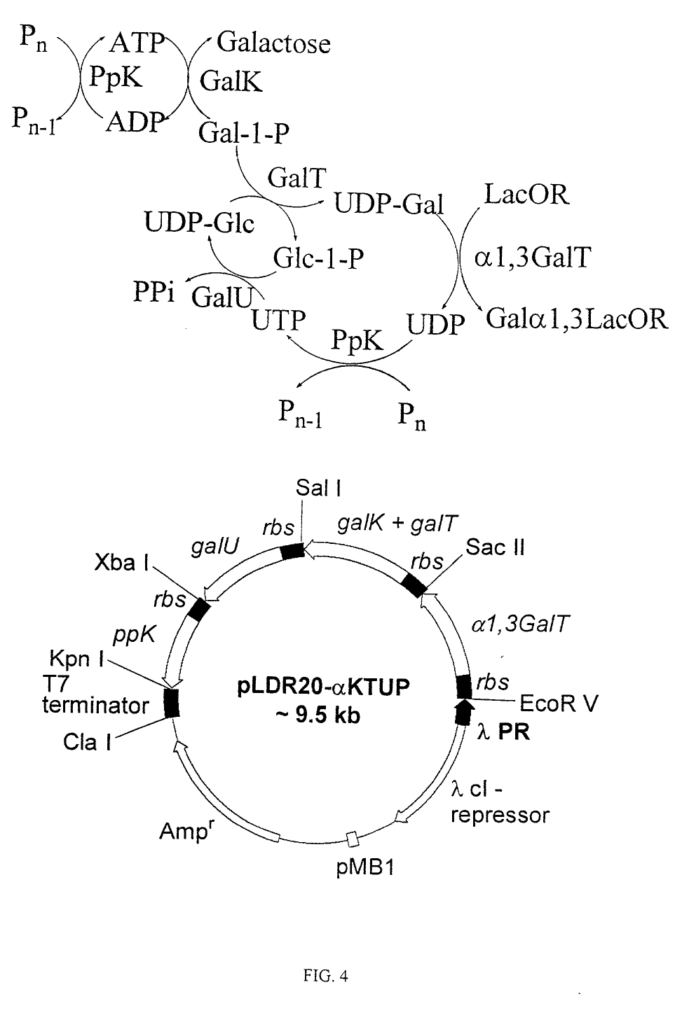

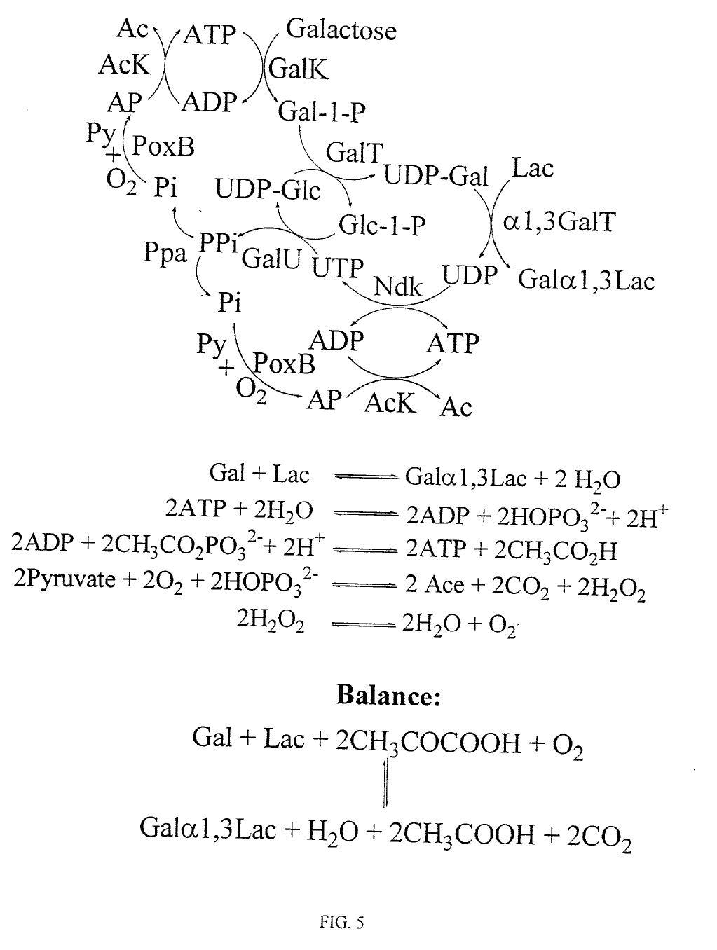

In certain embodiments, UDP-Gal is regenerated. Generally, galactose is provided to beads having enzymes capable of regenerating UDP-Gal immobilized thereon. Galactose is converted into Gal-1-P, which is subsequently converted to UDP-Gal. After the Gal of UDP-Gal is utilized by the glycosyltransferase, the resulting UDP is converted into UTP, which is subsequently converted into UDP-Glc. The UDP-Glc is then used to create UDP-Gal. Examples of enzymes used together to regenerate UDP-Gal include GalK, GaiT, and GalU. How these enzymes, along with bioenergetics and glycosyltransferase, complete the above tasks is exemplified in FIG. 1, FIG. 3, FIG. 4, FIG. 5, and FIG. 15.

Other enzymes may be used to regenerate UDP-Gal. For example, an epmierase, such as GalE, may be included with GLK, PGM, and GalU. Also, as shown in FIG. 15, the functions of sucrose synthase and GalE can be combined to regenerate UDP-Gal.

### B. UDP-GIc

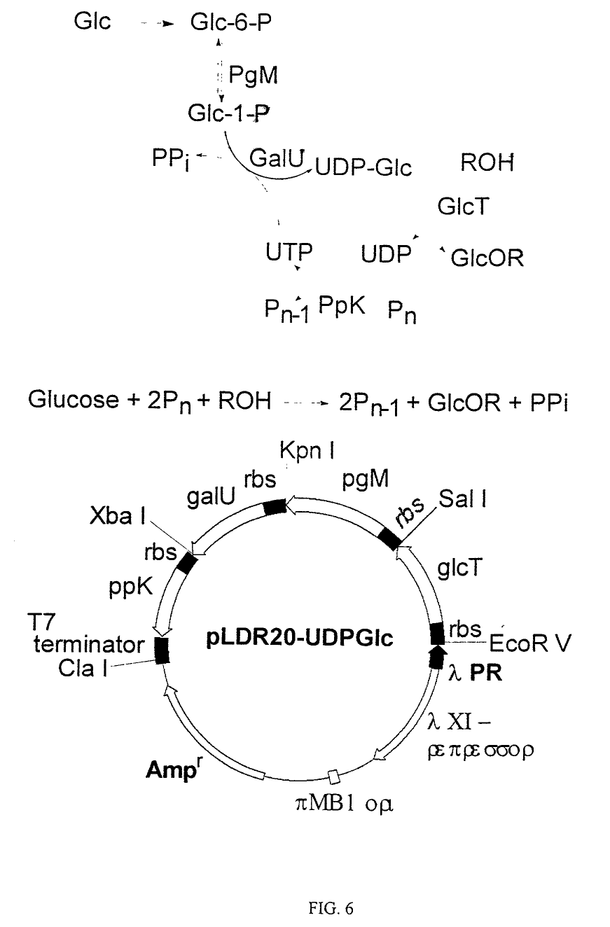

In other embodiments, UDP-Glc is regenerated. One method of regenerating UDP-Glc is through the glucose metabolism pathway as diagrammed in FIG. 6. UDP-Glc is regenerated by the combination of phosphoglucomutase (PGM), EC 5.4.2.2) (e.g. Leyva-Vazquez and Setlow, J. Bacteriol. 1994, 176: 3903-3910; Lu and Kleckner, Bacteriol. 1994, 176: 5847-5851; Pradel and Boquet, Res. Microbiol. 1991, 142: 37-45), glucose-1-phosphate uridyltransferase (GalU) and polyphosphate kinase (PpK). Since GalU acts on glucose-1-phosphate, glucokinase, which phosphorylates glucose, is also required in this system.

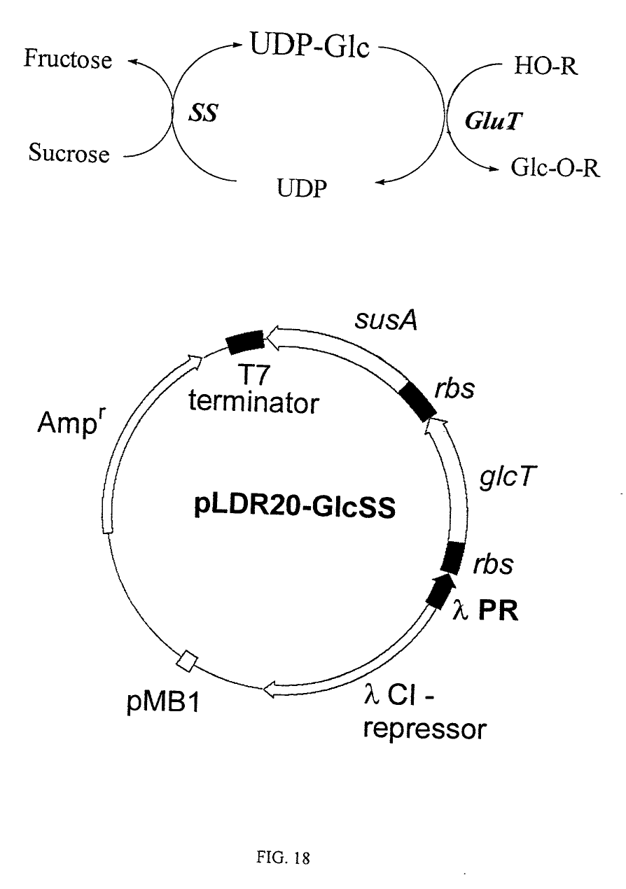

Other enzymes may be used to regenerate UDP-Glc. For example, an epimerase, such as GalE, may be included with GalK, GalT, and GalU. Also, sucrose synthase, which converts UDP to UDP-Glc directly with the consumption of sucrose can be utilized, as shown in FIG. 18.

### C. UDP-GlcNAc

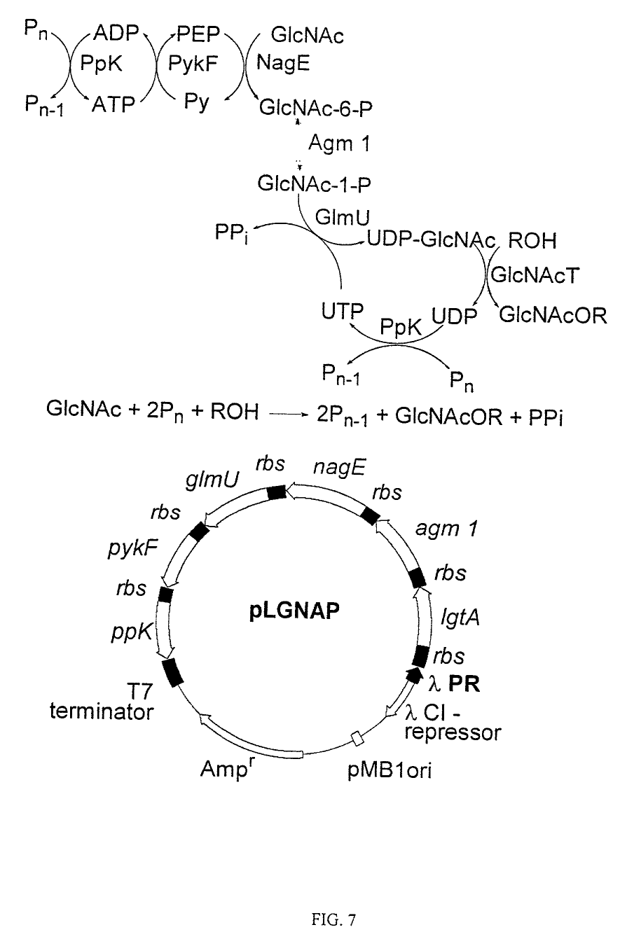

In other embodiments, UDP-GlcNAc is regenerated. One method of regenerating UDP-GlcNAc is diagrammed in FIG. 7. In this UDP-GlcNAc regeneration system, GlcNAc-1-phosphate uridyltransferase from Escherichia coli (e.g. glmU; Brown, K. et al., EMBO J. 1999, 18: 4096-4107; Gehring, A. M. et al., Biochemistry 1996, 35: 579-585; Mengin-Lecreulx and van Heijenoort, J. Bacteriol. 1993, 175: 6150-6157), N-acetylglucosamine permease from Vibrio furnissii (nagE; Yamano, N. et al., Biosci. Biotechnol. Biochem. 1997, 61: 1349-1353), N-acetylglucosarnine-phosphate mutase from Saccharomyces cerevisiae (agml; Mio, T. et al., J. Biol. Chem. 1999, 274: 424-429) are used to regenerate the sugar nucleotide. Also, used in this system are the gycosyltransferase β1,3GlcNAc transferase from Neisseria meningitidis (LgtA; Blixt, 0. et al., Glycobiology 1999, 9: 1061-1071) together with polyphosphate kinase and pyruvate kinase (ppK and pykF, respectively). In the system shown in FIG. 7, polyphosphate is the bioenergetic.

In a preferred embodiment, all of the enzymes of the UDP-GlcNAc regeneration system diagrammed in FIG. 7 are immobilized on the beads. These beads are particularly useful for UDP-GlcNAc regeneration and GlcNAcβ1,3LacOR synthesis. GlcNAcβ1,3LacOR is a core structure in α-Gal pentasaccharides (important for xenotransplantation research) and lipopolysaccharides on the membrane of Neisseria meningitidis.

### D. UDP-GalNAc

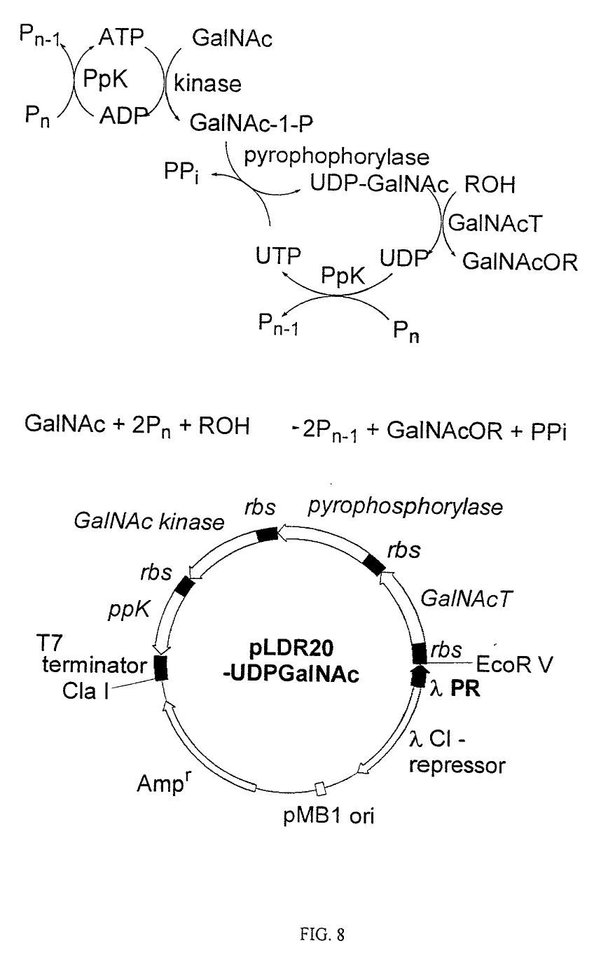

In other embodiments, UDP-GalNAc is regenerated. One method of regenerating UDP-GalNAc is diagrammed in FIG. 8. In this method, UDP-GalNAc is biosynthesized directly from GalNAc by a GalNAc-1 kinase and then by a pyrophosphorylase (uridyltransferase). This route is derived from a pathway for salvage of GalNAc generated by the degradation of glycosaminoglycans and glycoproteins in eukaryotes. One particularly useful utility of UDP-GalNAc reneration is, when coupled with a human UDP-GalNAc:2′-fucosylgalactoside-α-3-N-acetylgalactosaminyl transferase, human blood type A antigen is synthesized.

The first gene in the pathway, GalNAc-1-phosphate kinase, was first identified by Pastuszak and co-workers from pig kidney in 1996 (Pastuszak, I. et al., J. Biol. Chem. 1996, 271: 20776-20782). The enzyme shows high specificity for GalNAc over other N-acetylated and non-acetylated aminosugars. It is a monomeric, 50 kDa protein with a divalent metal requirement (5 MM Mg²⁺ optimal). The enzyme is most active with ATP as the high-energy phosphate donor. However, some activity is also detected with ITP, acetyl-phosphate and phosphoenolpyruvate (PEP). Significant GalNAc-1-P kinase activity has also been detected in human kidney and liver and the sequence of peptides from the GalNAc kinase have been reported (Pastuszak, I. et al., J. Biol. Chem. 1996, 271: 23653-23656). These peptides showed very high homology with the human galactokinase reported on chromosome 15 and in fact the authors, based on further biochemical evidence, reassigned this human kinase as GalNAc kinase.

The second enzyme of this pathway is UDP-GalNAc pyrophosphorylase. Purified to homogeneity by Szumilo and others (Szumilo, T. et al., J. Biol. Chem. 1996, 271: 13147-13154; Wang-Gillam, A. et al., J. Biol. Chem. 1998, 273: 27055-20757), the protein is 64 kDa by SDS-PAGE. The Km value for GalNAc-1-P was 0.29 mM and for GlcNAc-1-P was 1.1 mM. This indicates that at low concentrations, UDP-GlcNAc is the preferred substrate, however at 5 mM UDP-GalNAc is as effective as UDP-GlcNAc. The enzyme's pH optimum is between 8.5 and 8.9 and it requires a divalent metal for activity (Mn²⁺>Mg²⁺>Co²⁺).

As in the case of UDP-Gal and UDP-Glc co-regeneration beads, UDP-GalNAc can also be biosynthesized from UDP-GaLNAc by an epimerase, UDP-GlcNAc 4-epimerase (EC 5.1.3.7). The protein with this activity has been isolated from both prokaryotic and eukaryotic sources. Creuzenet and co-workers identified the wbpP gene encoding the UDP-GlcNAc 4-epimerase activity from A. aeruginosa (Creuzenet, C. et al., J. Biol. Chem. 2000, 275: 19060-19067). The proposed gene product shows a conserved nucleotide-binding-protein motif (GXXGXXG; SEQ ID NO: 1) and a catalytic triad (SYK) with E. coli UDP-Gal 4-epimerase (GalE), which provides an opportunity to identify and clone enzymes with this function from other prokaryotic sources based on sequence alignment and other bioinformatic methods.

### E. UDP-GIcA

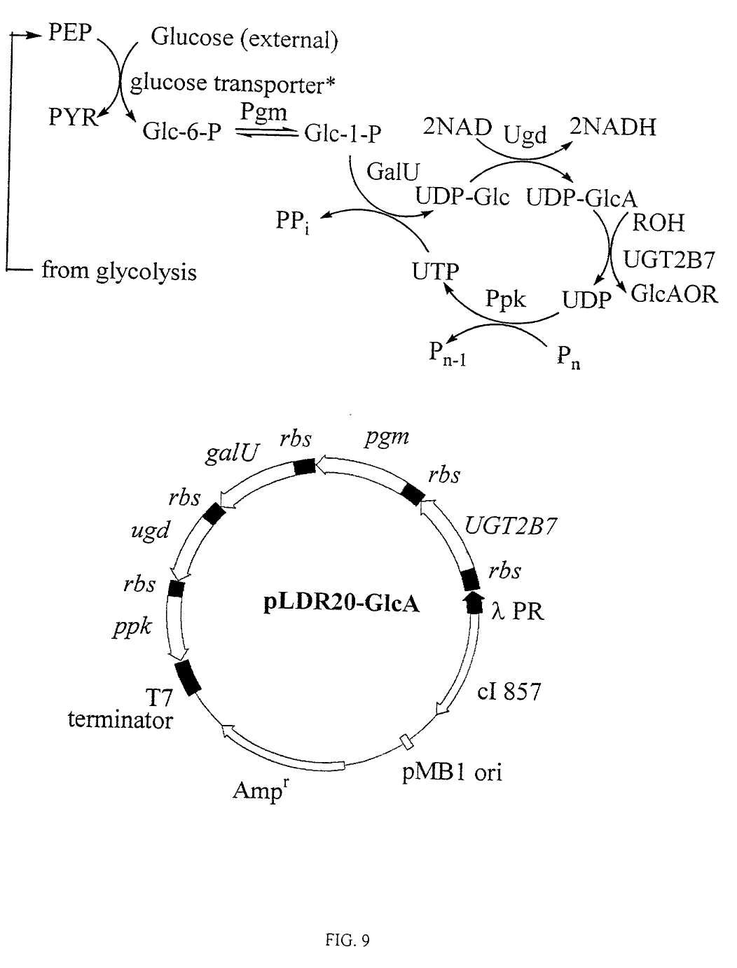

In other embodiments, UDP-GlcA is regenerated. One method of regenerating UDP-GlcA is diagrammed in FIG. 9. In all living systems, UDP-GlcA is synthesized from UDP-Glc by UDP-Glc 6-dehydrogenase (UDPGDH). This step is the control point to all the subsequent UDP-GlcA utilizing reactions. One equivalent of UDP-Glc is oxidized to one equivalent of UDP-GlcA with concomitant reduction of two equivalents of NAD⁺ to NADH. The UDP-GlcA regeneration system may be constructed by adding UDP-GlcA 6-dehydrogenase and substituting the gene of glucosyltransferase with a human UGT2B7 gene into the UDP-Glc regeneration system described in FIG. 6 to produce pLDR20-GlcA (See FIG. 9). The enzyme products of this system can then be immobilized on the bead. Also, the NAD⁺ co-factor can be provided to the bead.

UDP-Glc 6-dehydrogenase activity has been isolated from a variety of organisms. The enzyme has been cloned from both human and mouse (Spicer, A. P. et al., J. Biol. Chem. 1998, 273: 25117-25124), bovine kidney (Lind, T. et al., Glycobiology 1999, 9: 595-600), and prokaryotic organisms Sinorhizobium meliloti (Kereszt, A. et al., J. Bacteriol. 1998, 180: 5426-5431), E. coli K5 (De Luca, C. et al., Bioorg. Med. Chem. 1996, 4: 131-134), and Bacillus subtilis 168 (Pagni, M. et al., Microbiology 1999, 145: 1049-1053.). The gene for this enzyme is also present in the Chlorella virus PBCV-1 and has been found to produce a functional protein early in infection (Landsterin, D. et al., Virology 1998, 250: 388-396).

Although essentially any of these UDP-Glc 6-dehydrogenases may be used in the compositions and methods of the present invention, a preferred UDP-Glc 6-dehydrogenase is encoded by the ugd gene from E. coli K12 (43 kD)(De Luca et al., Bioorg Med Chem 1996, 4(1):131-41) because it does not contain internal restriction sites for the other enzymes used in the construction of the multiple enzyme vectors. This property greatly facilitates construction of a plasmid for cloning and overexpressing the enzyme, which facilitates the production of beads in accordance with the present invention.

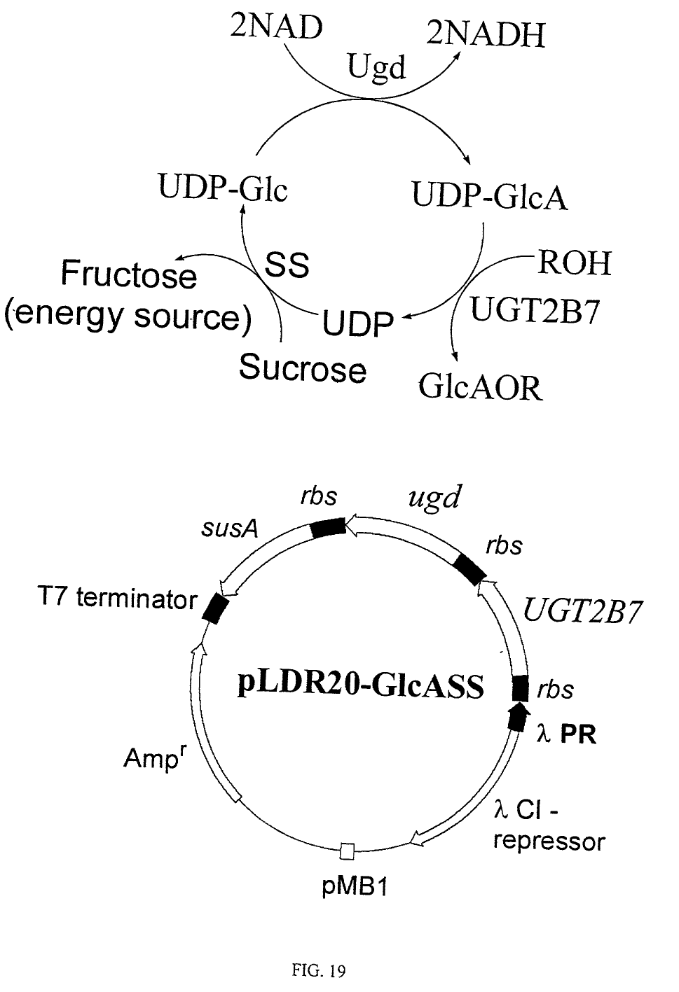

As in the case of producing UDP-Glc, UDP-GlcA can be regenerated or synthesized using sucrose synthase and UDP-Glc 6-dehydrogenase as shown in FIG. 19.

### F. CMP-NeuNAc

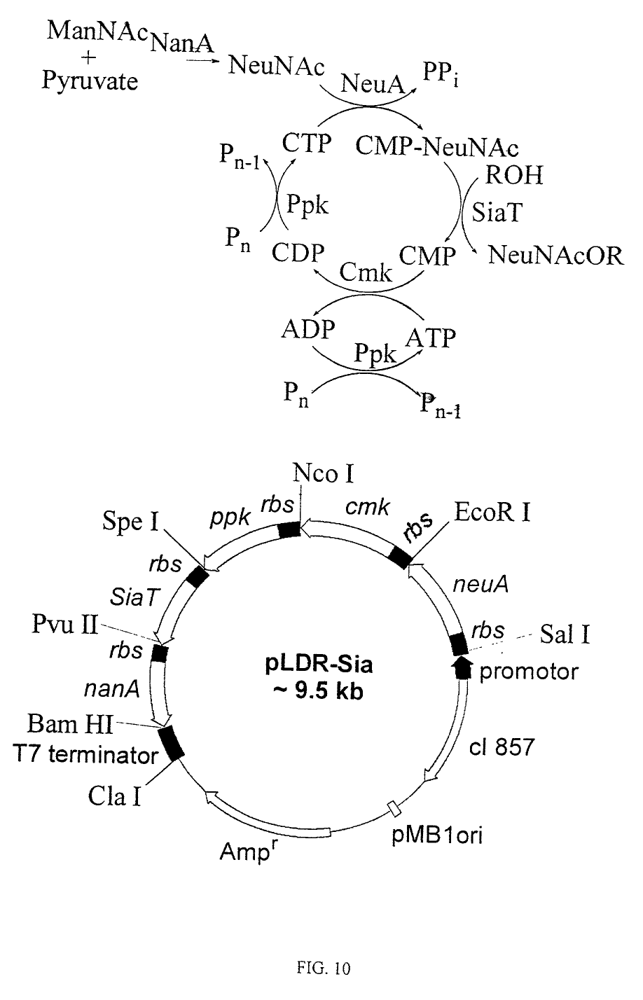

In other embodiments, CMP-NeuNAc is regenerated. One method of regenerating CMP-NeuNAc is diagrammed in FIG. 10. A particularly useful plasmid for the expression of enzymes necessary for the regeneration of CMP-NeuNAc is pLDR-Sia (FIG. 10). This plasmid encodes sialic acid aldolase (NanA), CMP-Neu NAC sythetase (NeuA), CMP kinase (Cmk), and polyphosphate kinase (Ppk) along with the glycosyltransferase α2,3 (or α2,6)-sialyltransferase (SiaT).

NeuAc aldolase (NanA, N-acetylneuraminate lyase, EC 4.1.3.3) catalyzes the reversible cleavage of NeuAc to form pyruvate and ManNAc. The enzyme has been exploited for the synthesis of NeuNAc or its derivatives (e.g. Murakami, M. et al., Carbohydr. Res. 1996, 280: 101-110; Mahmoudian, M. et al., Enzyme. Microb. Technol. 1997, 20: 393-400; Maru, I. et al., Carbohydr. Res, 1998, 306: 575-578; Aisaka, K. et al., Biochem. J. 1991, 276: 541-546; Walters, D. M. et al., J. Bacteriol. 1999: 181: 4526-4532; Lilley, G. G. et al., Protein Expr. Purif 1992, 3: 434-440). The E. coli NanA is a tetramer with an optimum pH around 7.7. The Kₘ for NeuNAc is 4.3 mM and pyruvate competitively inhibits the cleavage reaction. The enzyme belongs to the Schiff-base-forming Class I aldolases and X-ray crystallographic structure available (Aisaka, K. et al., Biochem. J. 1991, 276: 541-546; Uchida, Y. et al., J. Biochem. (Tokyo) 1984, 96: 507-522). When the E. coli gene encoding NanA was cloned into the pET15b vector downstream of the T7 promoter, the overexpressed protein consisted of more than 50% of the total cellular protein. About 30,000 units of active enzyme can be obtained from one liter of bacterial culture.

CMP-NeuNAc synthetase (NeuNAcS, N-Acetylneuraminic acid cytidylyltransferase, EC 2.7.7.43) catalyzes the formation of CMP-NeuNAc (Vann, W. F. et al., J. Biol Chem. 1987, 262: 17556-17562; Vionnet, J. et al., Glycobiology 1999, 9: 481-487; Munster, A. K. et al., Proc. Natl. Acad. Sci U.S.A. 1998, 95: 9140-9145). The enzyme purified from E. coli K1 requires Mg²⁺ or Mn²⁺ and exhibits optimal activity between pH 9.0 and 10. The apparent Kₘ for CTP and NeuNAc are 0.31 and 4 mM, respectively. The gene encoding NeuNAcS from E. coli serotype 07 K1 was isolated and overexpressed in E. coli W3110 with expression level up to 8-10% of the soluble E. coli protein. The over-expressed synthetase was purified to greater than 95% homogeneity and used directly for the synthesis of CMP-NeuNAc and derivatives (Shames, S. L. et al., Glycobiology 1991, 1: 187-191). Other researchers have also reported the enzymatic synthesis of CMP-NeuNAc using NeuNAcS (Aisaka, K. et al., Biochem. J. 1991, 276: 541-546; Kittelmann, M. et al., Appl. Microbiol. Biotechnol. 1995, 44: 59-67).

CMP kinase from E. coli is a monomeric protein of 225 amino acid residues. The protein exhibits little overall sequence similarity to other known NMP kinases. However, the residues involved in substrate binding and/or catalytic motif(s) were found to be conserved, and sequence comparison suggests a similar global structure found in adenylate kinases or several other CMP/UMP kinases (Bucurenci, N. et al., J. Biol. Chem. 1996, 271: 2856-2862; Briozzo, P. et al., Structure 1998, 6: 1517-1527). Substrate specificity studies shows that CMP kinase from E. coli is active with ATP, dATP, or GTP as donors and with CMP, dCMP, and arabinofuranosyl-CMP as acceptors (Bucurenci, N. et al., J. Biol. Chem. 1996, 271: 2856-2862; Briozzo, P. et al., Structure 1998, 6: 1517-1527).

In a preferred embodiment, all of the enzymes of the CMP-NeuNAc regeneration system diagrammed in FIG. 10 are immodbilized on the beads.

### G. GDP-Man

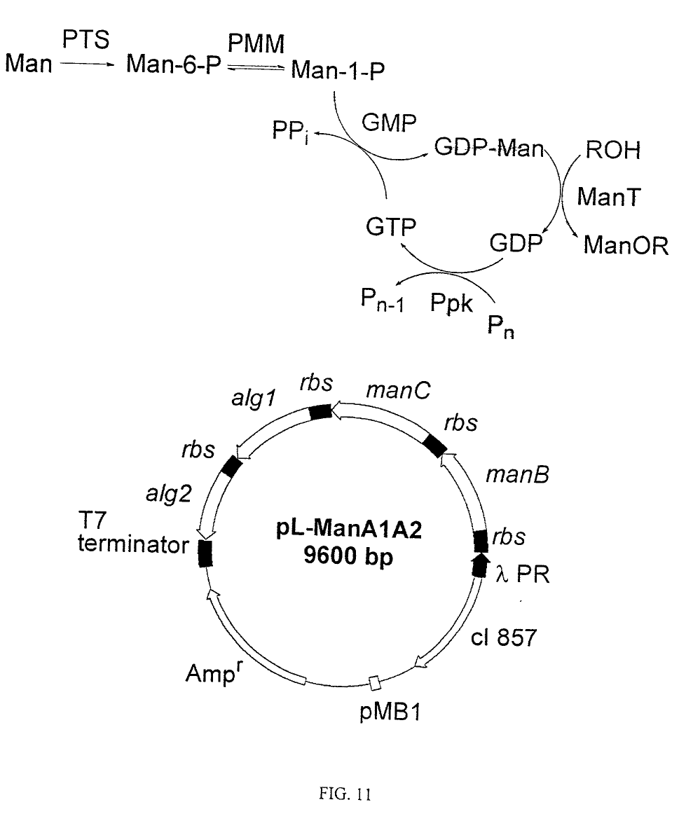

In other embodiments, GDP-Man is regenerated. One method of regenerating GDP-Man is diagrammed in FIG. 11. A particularly useful plasmid for the expression of enzymes necessary for the regeneration of GDP-Man is pL-ManA1A2 (FIG. 11).

The biosynthesis of GDP-mannose can start with mannose 6-phosphate which is automatically phosphorylated from mannose when transported into the cell via the PEP-dependent transporter system (PTS). In a preferred embodiment, phosphomannomutase (PMM, EC 5.4.2.8) and GDP-mannose pyrophosphorylase (GMP, EC 2.7.7.13), two key enzymes contributing to the pathway of GDP-mannose regeneration are co-immobilized on the bead along with a mannosyltransferase for the synthesis of mannose-terminated glycoconjugates (FIG. 11).

PMM catalyzes the interconversion of mannose-6-phosphate and mannose-1-phosphate. In the rfb gene cluster of E. coli 09 strain, rfbK was indicated to encode PMM and rfbM encodes GDP-mannose pyrophosphorylase (GMP, EC 2.7.7.13)(Marolda and Valvano, J. Bacteriol. 1993, 175: 148-158; Sugiyama, T. et al., Microbiology 1994, 140: 59-71; Jayaratne, P. et al., J. Bacteriol. 1994, 176: 3126-3139). In E. coli K-12 strain, there is a wca (cps) gene cluster comprising another pair of isogenes termed cpsG(manS) and cpsB(manC) encoding PMM and GMP, respectively. The cpsG(manS) and cpsB(manC) genes contribute to the production of both GDP-mannose and GDP-fucose (Aoyama, K. et al., ; Mol. Biol. Evol. 1994, 11: 829-838). The manB gene (1371 bp) encodes a predicted 50.5 kDa protein that requires Mg²⁺ or Mn²⁺ for activity (Zielinski, N. A. et al., J. Biol. Chem. 1991, 266: 9754-9763; Goldberg, J. B. et al., J. Bacteriol. 1993, 175: 1605-1611; Coyne, M. J. et al., J. Bacteriol. 1994, 176: 3500-3507; Ye, R. W. et al., J. Bacteriol. 1994, 176: 4851-4857). The crystal structure of the enzyme has been published (Regni, C. A. et al., Acta Crystallogr. D. Biol. Clystallogr. 2000, 56:761-762). The manC gene has 1437 bp encoding a 53.0 kDa protein which is also termed GTP:mannose 1-phosphate guanylyltransferase (EC 2.7.7.22) describing the reverse reaction.

### H. GDP-Fuc

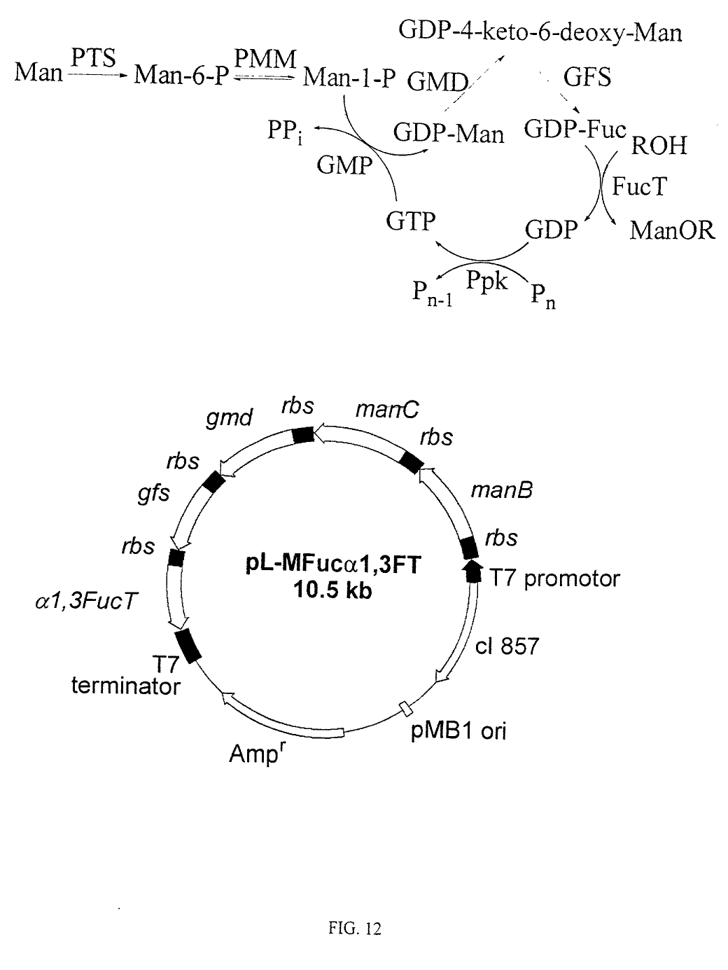

In other embodiments, GDP-Fuc is regenerated. One method of regenerating GDP-Fuc is diagrammed in FIG. 12. A particularly useful plasmid for the expression of enzymes necessary for the regeneration of GDP-Fuc is pL-Fucα1,3FT (FIG. 12).

The major pathway to generate GDP-fucose from GDP-mannose is present in both prokaryotes and eukaryotes. Two routes can be proposed. One of the routes carries out the formation of GDP-fucose from GDP-mannose in three steps using two enzymes. An alternate pathway is a two step procedure to form GDP-fucose from fucose (Pastuszak, I. et al., J. Biol. Chem. 1998, 273: 30165-30174). The first route is illustrated in FIG. 12. A GDP-Fuc regenerating bead can be easily obtained by modifying an existing GDP-Man regenerating bead by co-immobilizing GMD (GDP-D-mannose 4,6-dehydratase, EC 4.2.1.47) and a bifunctional GFS (GDP-L-fucose synthetase) or GMER (GDP-4-keto-6-deoxy-D-mannose epimerase/reductase) with existing GDP-Man regeneration enzymes on the bead and substituting a fucosyltransferase such as α1,3FucT (FIG. 12) for the mannosyltransferase.

Three steps are involved in the conversion of GDP-fucose from GDP-mannose including 4,6-dehydrogenation, 3,5-epimerization, and 4-reduction (Bonin, C. P. et al., Proc. Natl. Acad. Sci. USA 1997, 94: 2085-2090; Ohyama, C. et al., J. Biol Chem. 1998, 273: 14582-14587). The enzyme involved in the first step, GMD from E. coli, has been cloned, expressed and characterized. (Mattila et al., Glycobiology 2000 10(10): 1041-7; Andrianopoulos et al., J Bacteriol 1998 180(4):998-1001; Sturla et al., FEBS Lett 1997 412(l):126-30). Metal ion Ca²⁺ and Mg²⁺ are required for the enzyme activity (Sturla, L. et al., FEBS Lett. 1997, 412: 126-130). The gmd gene may be cloned by PCR from the wca (cps) gene cluster of E. coli K-12, which contains 1122 bp encoding a predicted 42.1 kDa protein (Stevenson G., et al., J Bacteriol 1996,178(16):4885-93).

E. coli protein GFF displays dual 3,5-epimerase and 4-reductase activities. Both epimerization and reduction reactions occur at the same site within a Ser-Tyr-Lys catalytic triad. The gene gfs (966 bp) found in E. coli K-12 encodes a 36.1 kDa protein (Rizzi, M. et al., Structure 1998, 6: 1453-1465).

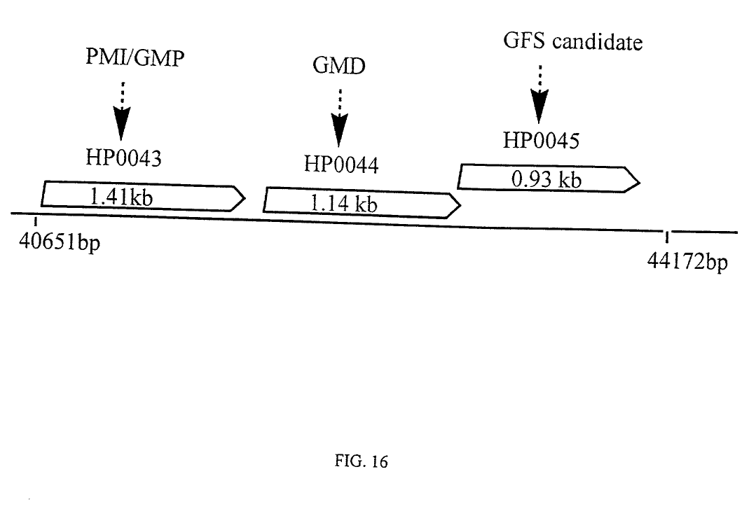

Upon examination of the genomic database of Helicobactor pylori (ATCC strain NO.700392), the inventors have identified four important enzymes (PMI, GMP, GMD, GFS) in the biosynthesis of GDP-Fuc in H. pylori that are encoded by a gene cluster. H. pylori can mimic the host surface antigens to escape elimination by the host immune system. For example, LPS 0-antigen of H. pylori commonly expresses human oncofetal antigens Lewis X and Lewis Y. Several fucosyltransferases have been identified and cloned from H. pylori (Martin et al., J. Biol. Chem. 1997, 272, 21349-21356; Wang et al., Mol. Microbiol. 1999, 31, 1265-1274; Rasko et al., J. Biol. Chem. 2000, 275, 4988-4994; Alm et al., Nature 1999, 397, 176-180; Wang et al., Microbiology 1999, 145, 3245-3253; Ge et al., J. Biol. Chem. 1997, 272, 21357-21363), however the source of donor GDP-fucose has not been previously determined. A BLAST gene search against the genome of H. pylori using sequences of GDP-fucose biosynthesis enzymes revealed a gene cluster (40651nt to 44172nt, HP0043, HP0044, HP0045 in FIG. 16) putatively responsible for GDP-fucose biosynthesis.

It is common that genes for the synthesis of certain sugar nucleotides are generally clustered together within bacterial genomes. HP0043 and HP0044 have been identified as putative PMI/GMP and GMD. In the genomic database, HP0045 is predicted as a nodulation protein K in H. pylori strain 26695, and a sugar biosynthesis gene in H. pylori strain J99. From the protein sequence comparison, the inventors determined that HP0045 has 37% sequence identity and 57% similarity with both GFSs of E. coli K12 (accession no 8569682) and Y. pseudotuberculosis (accession no CAB63301). The multiple sequence alignment shows that HP0045 contains many conserved residues, which form characteristic motifs. The conserved Ser-Tyr-Lys catalytic triad in GFS of E. coli is located at S107 and Y136, K140. This triad is involved in catalyzing the reaction (Y136, K140) and interacting with the substrate to stabilize its conformation (S107, K140). Other residues related to NADP(H) binding are also found in HB0045, such as Leu 41, Ala 63. The characteristic GXXGXXG motif is observed at the N-terminus. In addition, GDP-sugar binding sites (Val 180, Leul84, Trp 202, etc.), phosphate binding sites (Lys 283, Arg209, etc), the 4-keto-sugar interaction sites (Ser107, Ser108, Cys109, Asnl65, etc.) can be found in HP0045 (Somers et al., Structure 1998, 6, 1601-6012). This analysis strongly suggests that HP0045 is a GFS gene in the GDP-fucose biosynthesis gene cluster. On the basis of gene similarity, this H. pylori GFS can be classified into the short-chain dehydrogenase/reductase family that catalyzes two distinct reactions at the same active site.

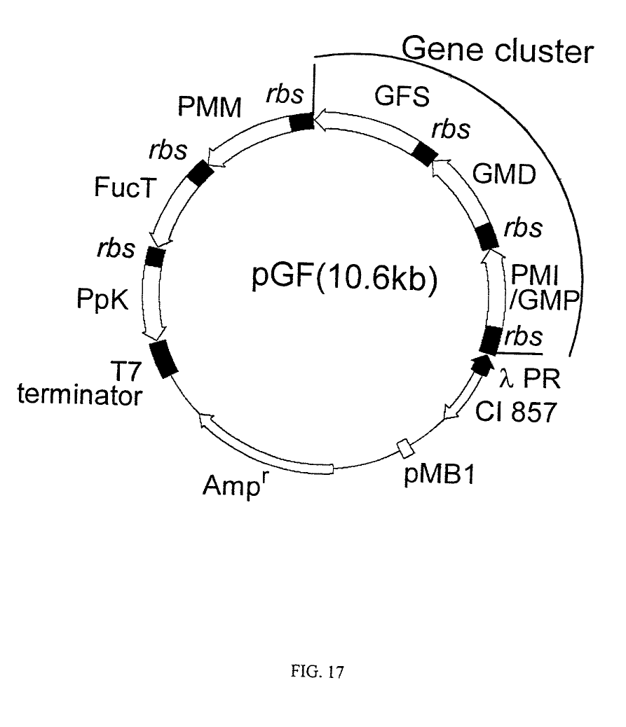

Based on the putative GDP-Fuc gene cluster in H. pylori, the construction of GDP-Fuc regeneration superbug is simplified. This gene cluster (3.5 kb) may be ligated in tandem with another three genes (PMM, PpK and FucT) to construct a recombinant plasmid. The plasmid for the synthesis of fucosylated glycoconjugates using the gene cluster is shown in FIG. 17. This plasmid can be used to facilitates the production of beads in accordance with the present invention.

### Epimerases

In certain embodiments, it may be useful to use an epimerase. For example, UDP-Gal and UDP-Glc can actually be inter-converted by UDP-galactose-epimerase (GalE) (Wilson and Hogness, J. Biol. Chem. 1964, 239: 2469-2481). Therefore, UDP-Gal and UDP-Glc can be co-regenerated by a bead having appropriate enzymes immobilized thereon through either galactose metabolic pathway (UDP-Gal regeneration) or glucose metabolic pathway (UDP-Glc regeneration). As long as GalE is immobilized on the bead, any of the systems illustrated in FIGS. 1, 3, 4, 5, 15 and 18 can be used for the regeneration of sugar-nucleotide donor for any glucosyltransferase or galactosyltranferase.

Examples of other epimerases that may be used in conjunction with the present invention include GlcNAc- 2-epimerase (GlcNAc; mannose), UDP GlcNAc -2-epimerase (UDP-ManNAc; UDP GlcNAc-2), and UDP GlcNAc- 4-epimerase (UDP GalNAc; UDP GlcNAc-4).

For each of the enzymes, suitable concentrations of substrate can be determined for various quantities of enzyme based upon a variety of parameters, including reaction product.

### Glycosyltransferases

An important aspect of the present invention is the transfer of the sugar moiety from the sugar nucleotide to an acceptor saccharide molecule. This process is carried out by a group of proteins known as glycosyltransferases. Essentially any glycosyltransferase may be used in conjunction with the compositions and methods of the present invention. A great number of glycosyltransferases are known and an extensive list of glycosyltransferases is provided in EP 0870841. A further source of glycosyltransferases, including source organism, EC#, GenBank/GenPept Accession Nos., SwissProt Accession No., and 3D structures, can be found at http://afmb.cnrs-mrs.fr/˜pedro/CAZY/gtf.html (Pedro Coutinho, Glycosyltransferase Families (last updated Nov. 17, 2000)).

The glycosyltransferase chosen is preferably specific to the sugar nucleotide that is regenerated by the sugar nucleotide regeneration enzymes immobilized on the bead. In preferred embodiments, the glycosyltransferase is co-immobilized on the beads along with the sugar nucleotide-regenerating enzymes. Alternatively, the glycosyltransferase can be supplied to the bead in solution, co-immobilized on the same bead, or may be immobilized on a separate bead or a separate population of beads.

Glycosyltransferases typically display specificity in regards to the donor saccharide molecule. Therefore, it is convenient to group them based on their donor specificity.

For each of the enzymes, suitable concentrations of substrate can be determined for various quantities of enzyme based upon a variety of parameters, including reaction product.

### A. Gal

A large number of glycosyltransferases that transfer galactose (galactosyltransferase) are known. Breton et al. provides an extensive list of prokaryotic and eukaryotic galactosyltransferases and is incorporated herein by reference (J. Biochem. 1998, 123:1000-1009). Another list can be found at http://stanxterm.aecom.yu.edu/glyc-T/galt.htm (visited Nov. 21, 2000). Galalactosyltransferases include α1,2 galactosyltransferases, such as Gma12p from yeast (Genbank Acc. No. Z30917), α1,3 galactosyltransferases , such as GGTA1 from mouse (Genbank Acc. No. M26925), β1,4 galactosyltransferases, such as GalT-I from human (Genbank Acc. No. X55415), and ceramide galactosyltransferases, such as CGT from Man (Genbank Acc. No. U30930). Galactosyltransferases that transfer galactose from UDP-Gal to an acceptor molecule include α1,3GalT, β1,4GalT (LgtB), and α1,4GalT (LgtC).

### B. Glc

Glycosyltransferases that transfer the glucose to an acceptor molecule are known as glucosyltransferases. Examples of glucosyltransferases include LgtF, Alg5, and DUGT (Heesen et al., (1994) Eur. J. Biochem. 224:71-79; Parker et al., (1995) EMBO J 14:1294-1303).

### C. GlcNAc

Glycosyltransferases that transfer the N-acetylglucosamine to an acceptor molecule are known as N-acetylglucosaminyl transferases. A number of N-acetylglucosaminyl transferases are known in the art and include LgtA (β1,3GlcNAc). A list of N-acetylglucosaminyl transferases can be found at http://www.vei.co.uk/TGN/glcnac.htm (lain Wilson (May 24, 1996)) and http://stanxterm.aecom.yu.edu/glyc-T/gnt.htm (visited Nov. 21, 2000). N-acetylglucosaminyl transferases include 1,2-N-acetylglucosaminyltransferases, such as MGAT1 from human (Genbank Acc. No. M55621), β1,4-N-acetylglucosaminyltransferases, such as GnT-III from human (Genbank Acc. No. D13789), and β1,6-N-acetylglucosaminyltransferases, such as GnT-V from human (Genbank Acc. No. D17716).

### D. GalNAc

Glycosyltransferases that transfer the N-acetylgalactosamine to an acceptor molecule are known as N-acetylgalactosaminyl transferases. A number of N-acetylgalactosaminyl transferases are known and include UDP-GalNAc:2′-fucosylgalactoside-α-3-N-acetylgalactosaminyl transferase. A list of N-acetylgalactosaminyl transferases can be found at http://www.vei.co.uk/TGN/galnac.htm (lain Wilson (May 24, 1996)). N-acetylgalactosaminyl transferases include α1,3-N-acetylgalactosaminyl transferases (blood group A)(Genbank Acc. No. J05175), β1,4-N-acetylgalactosaminyl transferases (Genbank Acc. Nos. M83651, L25885, U18975, and D17809), CT antigen transferases (Genbank Acc. No. L30104), and polypeptide GalNAc transferases (Genbank Acc. Nos. L17437, X85018, and D85389).

### E. GIcA

Glycosyltransferases that transfer glucuronic acid to an acceptor molecule are known as glucuronyltransferases. A list of glucuronyltransferases can be found at http://www.vei.co.uk/TGN/glcuron.htm (lain Wilson (May 24, 1996)). Examples of glucuronyltransferases include UGT1A (Swissprot Acc. No. P22309), UGT1B (Swissprot Acc. No. P36509), UGT1C (Swissprot Acc. No. P35503), UGT1D (Swissprot Acc. No. P22310), and UGT1F (Swissprot Acc. No. P19224). An example of a glucuronyltransferase that recognizes UDP-GlcA to transfer glucuronic acid to an acceptor molecule is UGT2B7.

### F. NeuNAc

Sialyltransferases are glycosyltransferases that transfer the N-acetylneuraminic acid to an acceptor. A number of sialyltransferases, including SiaT 0160 (EC 2.4.99.1), are known in the art. (lain Wilson, http://www.vei.co.uk/TGN/neuac.htm (May 24, 1996)). Sialyltransferases include α2,3-sialyltransferases, such as those desribed by Genbank Acc. Nos. X80503, L13972, X76989, X76988, L23768, X74570, and L23767, α2,6-sialyltransferases, such as those described by Genbank Acc. Nos. X75558, A17362, D16106, X74946, X77775, and L29554, and α2,8-sialyltransferases such as those described by Genbank Acc. Nos. D26360, X84235, U33551, L13445, X80502, and L41680.

Microbial oc-2,3-sialyltransferase from N. meningitidis consists of 371 amino acids (Gilbert, M. et al., J. Biol. Chem., 1996, 271: 28271-28276), showing unusual acceptor specificity in that it could use α- and β-terminal Gal residues as acceptors. In addition, (β1,4)-linked and (α1,3)-linked terminal Gal also serve as acceptors. Topology analysis shows that the N-terminal 6 to 24 residues is a non-cleavable signal sequence acting as a membrane anchor, with the catalytic domain facing the periplasmic space. In a preferred embodiment, the non-cleavable signal sequence is replaced by a cleavable signal sequence (pelB leader sequence in pET22b(+) vector, Novagen) so that the cloned sialyltransferase will be exported into periplasmic space with correct folding.

Microbial α2,6SiaT (SiaT 0160, EC 2.4.99. 1) has been purified from a marine bacterium Photobacterium damsela (Yamamoto, T. et al., J. Biochem. (Tokyo) 1996, 120: 104-110). The deduced amino acid sequence does not contain the sialyl binding motif and had no significant similarity to mammalian sialyltransferases. A homologous sequence of SiaT 0160 exists in Pasteurella multocida PM70, with an overall identity of 35% and similarity of 53%. The predicted protein has 412 residues and an N-terminal hydrophobic region that possibly functions as a signal sequence as the one in SiaT 0160. Therefore, the putative protein might be a potential α2,6SiaT. The putative ORF may be cloned, expressed and characterized to determine if it has α2,6SiaT activity.

### G. Man

Many mannosyltransferases are known in the art (Iain Wilson, http://www.vei.co.uk/TGN/man.htm (May 24, 1996)). Mannosyltransferases include α1,2-mannosyltransferases such as those described by Genbank Acc. Nos. M81110, X62647, and X89263, α1,3-mannosyltransferases such as that described by Genbank Acc. No. X87947, β1,4-mannosyltransferases such as that described by Genbank Acc. No. J05416, Ochl (Genbank Acc. No. D11095), Mnnl (Genbank Acc. No. L23753), MnnlO (Genbank Acc. No. L42540) Dpml (Genbank Acc. No. J04184), and Dol-P-Man:protein mannosyltransferases such as PMT1 (Genbank Acc. No. L19169). Mannosyltransferases that transfer the mannose moiety from GDP-Man to an acceptor saccharide molecule include Algl (μ1,4-linkage)(Takahashi, T. et al., Glycobiology 2000, 10, 321-327) and Alg2 (α1,3-or α1,6-transferase)( Jackson, B. J. et al., Arch. Biochem. Biophys. 1989, 272: 203-209; Yamazaki, H. et al., Gene 1998, 221: 79-84).

### H. Fuc

A list of known fucosyltransferases is provided at http://www.vei.co.uk/TGN/fuc.htm (lain Wilson, (May 24, 1996)) and http://stanxterm.aecom.yu.edu/glyc-T/fut.htrnl (visited Nov. 21, 2000). Glycosyltransferases that transfer the fucose from GDP-Fuc to an acceptor saccharide molecule include α1,3-FucT (Rizzi, M. et al., Structure 1998, 6: 1453-1465; Martin, S. L. et al., J. Biol. Chem. 1997, 272: 21349-21356), α1,2-FucT (Wang, G. et al., Mol. Microbiol. 1999, 31: 1265-1274), and α1,3/4-FucT (Wang, 1999). Other fucosyltransferases include α1,2-fucosyltransferases, such as those described by Genbank Acc. Nos. m35531, S79196, X91269 and U17894, α1,3/4-fucosyltransferases, such as those described by Genbank Acc. Nos. X87810, X53578, U27326, α1,3-fucosyltransferases, such as those described by Genbank Acc. Nos. M58596, U58860, M81485, L01698, and U08112, and α1,6-fucosyltransferases, such as that described by Genbank Acc. No. D86723.

### Solid Supports (Beads)

The solid support of the present invention provides the substance onto which one or more of the enzyme(s) of the glycoconjugate synthesis system can be immobilized. Preferably, the enzyme(s) utilized in the present invention are in proximity to each other, such as in a reaction vessel. Immobilization of the enzymes onto a solid support greatly facilitate the achievement of this proximity between enzyme(s). As such, the solid support is an important component of the present invention because the immobilization of the enzymes lends several of the advantages of the present invention, including increased enzyme stability, ease of separation, and generation of glycoconjugates in a cell-free environment. Preferably, the solid support comprises one or more microspherical beads, commonly referred to as “beads,” “superbeads,” and “microspheres.” Alternatively, though, the solid support can take a variety of forms, including strands, sheets, and plates.

The beads of the present invention can take a variety of sizes, but preferably are on the order of 10 to 2000 μm in diameter. More preferred are beads having a diameter of 10 to 500 μm. Particularly preferred are beads having a diameter between 45 and 165 μm. All beads used in any given reaction can have the same diameter, or approximately so, or different bead populations can have different diameters, which can facilitate later separation of the beads into their respective populations, if desired.

A variety of materials can be used as the base of the beads of the present invention. The choice of base material is largely dependent on the need for beads that are small in size, able to withstand elevated temperatures and vigorous shaking and mixing, and able to allow immobilization of the appropriate enzymes. For example, the base can comprise agarose, methacrylate, cellulose, polystyrene, polystyrene coated ferric oxide, or silica coated ferric oxide.

A variety of binding systems can be incorporated into the design of the beads to facilitate the immobilization of appropriately tagged enzymes. For example, a metal ion, typically Ni²⁺, immobilized on the bead can bind proteins having several consecutive Histidine residues. Thus, beads having Ni²⁺ chelated in the bead or immobilized thereon are able to bind such proteins. Other binding systems utilize the affinity between various binding pairs to achieve a similar result, and can be incorporated into the beads of the present invention. For example, bindings systems such as ones utilizing glutathione-S-transferase fusion proteins and immobilized glutathione, monoclonal or polyclonal antibodies and an antigen, immunoglobulins and Protein A or Protein G, avidin and biotin, or fusion proteins having a domain capable of binding the base material of the bead, such as a cellulose binding domain, can be utilized. In each instance, the bead must be engineered to contain one component of the binding system and the enzymes must be engineered to contain the other. For example, the beads can be engineered to contain avidin and the enzymes can be biotinylated to create an avidin-biotin binding system.

A single enzyme type can be immobilized on a bead or population of beads, or multiple types of enzymes can be immobilized on the beads. The bead need only contain a member of a binding pair that allows binding to the enzyme(s) of interest, which needs to contain the other member of the binding pair. A single binding pair can be used, or several different types can be used with each type of binding pair representing one or more types of enzymes.

Preferably, after the enzymes are immobilized on the solid support, or beads, the activity of the enzymes is confirmed prior to using the beads in a reaction according to the present invention. Preferred methods of confirming the activity of various preferred enzymes are detailed in the Examples.

In a preferred embodiment, nickel-NTA beads are used as the solid support. The nickel-NTA beads utilize chelated Ni²⁺ to bind enzymes containing polyhistidine tags, e.g., regions containing several consecutive histidine residues. In this embodiment, the beads are engineered to contain the chelated Ni²⁺ and the enzymes are engineered to contain an appropriate histidine tag. A hexo-histidine tag is preferred, but as few as three or four consecutive Histidine residues can be utilized. The histidine tag is obtained by inserting the gene for the enzyme into an appropriate vector, such as the pET system from Novagen, Madison, Wiss. The various enzymes necessary for carrying out the glycoconjugate synthesis can be obtained by cloning and overexpression of the genes for the hexo-histidine tagged enzymes along the sugar nucleotide biosynthetic pathway in a single recombinant bacterium or a plurality of different recombinant bacteria.

The nickel-NTA beads offer several advantages, including convenient and easy immobilization of appropriate enzymes onto the bead surface and rechargeability. The method of immobilizing necessary enzymes to the nickel-NTA beads is relatively simple. Due to the nickel content of the beads, histidine residues bind readily to the bead. Therefore, for simple immobilization, the enzymes to be immobilized on the beads containing the hexo-histidine tags at the N-terminus are simply passed over the beads or incubated in a reaction vessel with the beads. When passed over the nickel-NTA beads, the enzymes of interest, i.e., the enzymes containing the histidine tag, bind to the beads via the interaction between the Histidine tags and the chelated Ni²⁺ on the beads, thereby accomplishing a one-step purification and immobilization process.

Another advantage of using nickel-NTA beads as the solid support is their ability to be recharged. If the ability of the beads to bind the Histidine-containing enzymes has diminished, the beads can be recharged with Ni²⁺ simply by incubating the beads with a nickel salt, such as nickelous sulfate, nickelous chloride, nickelous carbonate, or nickelous acetate.

If bacteria are utilized as the source of the enzymes, the enzyme can be obtained from a cell lysate. A lysate can be obtained by lysing the cells in a simple buffered detergent solution containing enzymes for the breakdown of the cell membrane and nucleic acids, such as a solution of 20 mM Tris-HCl, pH 7.9, 1% Triton X-100, 200 ug/mL lysozyme, 2 ug/mL Dnasel. Essentially any buffer having a pH of between about 6 and about 8 can be utilized. Also, a buffer without lysozyme and/or Dnabel can also be utilized if other methods of disrupting the cell membrane are employed, such as French press or Sonication Methods known in the art. One familiar in the art will appreciate the basic task of obtaining a cell lysate, and will understand that several variations of such a solution will result in a suitable cell lysate. If multiple recombinant bacteria are utilized, the lysates of the several organisms are preferably combined and processed together. Alternatively, each could be processed and immobilized independently. Once obtained, the lysates are then assayed for activity of each individual enzyme of interest. Preferably, the enzyme activity is expressed in units that are equivalent to the amount of enzyme that catalyzes the production of 1 μmole product per minute at 24° C. Next, a cell lysate mixture is prepared that contains equal activity for all enzymes. This can be performed by combining lysates of multiple recombinant bacteria in a relative volume ratio based on the individual activity levels of each enzyme. Finally, the lysate mixture is incubated with the nickel-NTA beads under suitable conditions to promote binding to the beads. Typically, a 30 minute incubation at room temperature with low speed shaking (e.g., approximately 100 rpm) is acceptable. Recombinant-enzyme-bound beads are subsequently washed twice. For each wash, the container holding the beads is inverted or shaken to mix thoroughly, and then spun in a centrifuge at a speed sufficient to pellet the beads. The beads can then be separated by aspirating the supernatant. Alternatively, the beads can be separated by simple filtration with buffer containing Tris-HCl (20 mM, pH 8.0) and NaCL (0.5 M). Following the wash step, the beads are ready for addition to a reaction mixture.

### Plasmids

One method of producing the enzymes utilized in the present invention is through vectors such as plasmids, phage, phagemids, viruses, artificial chromosomes and the like. The type of vector to be used often will be dependent on the type of solid support to be engineered. The vector is placed into an organism for subsequent replication and production of the enzyme(s). Preferably, the vector is capable of replicating autonomously within the organism being utilized. However, the vector also may integrate into the host's genome and replicate along with the rest of the host's genome.

Preferred vectors are expression vectors. Particularly preferred vectors are expression vectors that contain coding regions that facilitate subsequent immobilization of the enzyme onto the solid support. Expression vectors contain a promoter that may be operably linked to a coding region. A gene or coding region is operably linked to a promoter when transcription of the gene initiates from the promoter. More than one gene may be operably linked to a single promoter. In preferred embodiments, at least one nucleotide regenerating enzyme gene or at least one glycosyltransferase is operably linked to the same promoter, and the vector allows for easy incorporation of a functional region, such as a poly-Histidine region, that facilitates immobilization.

Expression vectors that may be used include, but are not limited to, pUC19 (Gene, 33: 103 (1985)), pBluescript II SK+(Stratagene, La Jolla), the pET System (NOVAGEN, Madison, Wiss.), pLDR20 (ATCC 87205), pBTrp2, pBTacl, pBTac2 (Boehringer Mannheim Colo.), pKYP10 (Japanese Published Unexamined Patent Application No. 110600/83), pKYP200 (Agric. Biol. Chem., 48: 669 (1984)), pLSAl (Agric. Biol. Chem., 53: 277 (1989)), pGELl (Proc. Natl. Acad. Sci. USA, 82: 4306 (1985)), pSTV28 (manufactured by Takara Shuzo Co., Ltd.), pPAl (Japanese Published Unexamined Patent Application No. 233798/88), and pCG11 (Japanese Examined Patent Application No. 91827/94). When a yeast strain is used as the host, examples of expression vectors that may be used includes YEpl3 (ATCC 37115), YEp24 (ATCC 37051), and YCp5O (ATCC 37419).

Essentially any promoter may be used as long as it can be expressed in the organism being utilized. A preferred promoter for E. coli is the λ P_{R} promoter. In the presence of the product of the λ C₁ repressor gene, transcription from the λ P_{R} promoter may be controlled. At temperatures below 37° C. the repressor is bound to the P_{R} promoter and transcription does not occur. At temperatures above 37° C. the repressor is released from the P_{R} promoter and transcription initiates. Thus, by growing the organism containing the vector at 37° C. or above, the genes are expressed.

When the organism is a yeast cell, any promoter expressed in the yeast strain host can be used. Examples include gal 1 promoter, gal 10 promoter, heat shock protein promoter, MF α1 promoter and CUP 1 promoter.

A ribosome-binding sequence (RBS) (prokaryotes) or an internal ribosome entry site (IRES) (eukaryotes) may be operably linked to the gene. The RBS or IRES is operably linked to the gene when it directs proper translation of the protein encoded by the gene. It is preferred that the RBS or IRES is positioned for optimal translation of the linked coding region (for example, 6 to 18 bases from the initiation codon. In vectors containing more than one gene, it is preferred that each coding region is operably linked to an RBS or IRES. A preferred RBS is AGAAGGAG (SEQ ID No. 2).

The gene or genes may also be operably linked to a transcription terminator sequence. A preferred terminator sequence is the T7 terminator (pET15b Vector System, Novagen, Madison, Wiss., 2000 Catalog).

The coding region of the gene may be altered prior to insertion into or within the expression vector. Alterations include deletions, additions, and substitutions. For example, a coding region that provides a functional region, such as a poly-Histidine sequence, that facilitates immobilization may be incorporated into the gene. When alterations are made, it is preferred that the alteration maintains the desired enzymatic function or specificity of the enzyme. However, in certain embodiments, it may be desired to alter the specificity of the enzyme. For example, one may wish to alter the sugar-nucleotide binding region of the enzyme such that the sugar-nucleotide specificity of the enzyme is changed.

When a heterologous gene is to be introduced into an organism that does not naturally encode the gene, optimal expression of the gene may require alteration of the codons to better match the codon usage of the host organism. The codon usage of different organisms is well known in the art.

The coding region also may be altered to ease the purification or immobilization. An example of such an alteration is the addition of a “tag” to the protein. Examples of tags include FLAG, polyhistidine, biotin, T7, S-protein, and GST (Novagen; pET system). In a preferred embodiment, the gene is altered to contain a hexo-histidine tag in the N-terminus. In this embodiment, the enzyme(s) may be purified and immobilized onto the solid support in one step by exposing the enzyme(s) to Ni²⁺ beads.

In other embodiments, the coding regions of two or more enzymes are linked to create a fusion protein. In preferred embodiments, a glycosyltransferase is fused with a corresponding epimerase (Chen et al., J Biol Chem 2000, 275(41):31594-31600). This fusion protein can be engineered to contain a functional region, such as a poly-Histidine sequence, that facilitates immobilization. This allows the fusion protein to be immobilized on the bead in an identical manner to simple, non-fusion proteins.

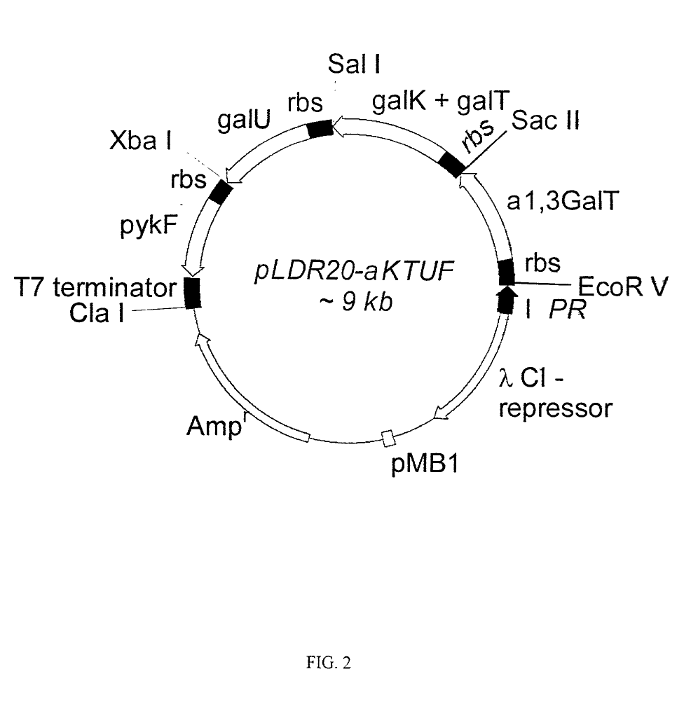

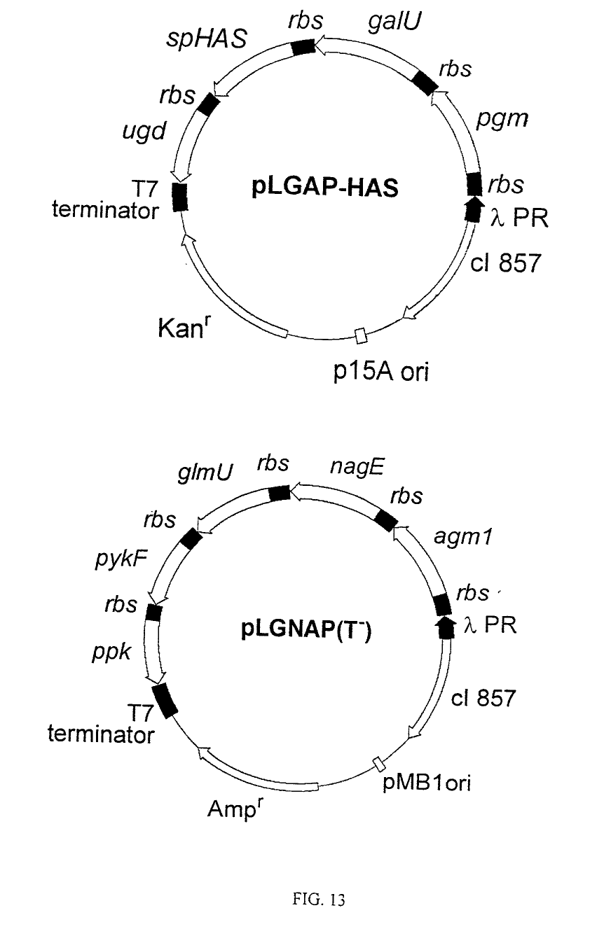

In further preferred embodiments, the expression vector of the present invention comprises at least one gene encoding a sugar-nucleotide regenerating enzyme and at least one glycosyltransferase, with each gene having a functional region that facilitates immobilization, such as a poly-Histidine region. The plasmid may also encode one or more enzymes that facilitate the catalysis of a bioenergetic. Preferred plasmids of the present invention include pLDR20-αKTUF (FIG. 2), pLDR20-αKTUN (FIG. 3), pLDR20-αKTUP (FIG. 4), pLDR20-UDPGlc (FIG. 6), pLGNAP (FIG. 7), pLDR20-UDPGalNAc (FIG. 8), pLDR20-GlcA (FIG. 9), pLGAP-HAS (FIG. 13), pLGNAP(T) (FIG. 13), pLDR-Sia (FIG. 10), pL-ManAlA2 (FIG. 11), pL-Mfucα1,3FT (FIG. 12), pLDR20-αES (FIG. 15), and pGF (FIG. 17) and plasmids constructed for individual enzymes involved in the glycoconjugate or sugar nucleotide synthesis and those for any combination of these enzymes.

### Organisms

To produce the enzymes of interest, the vectors described above can be used with a variety of organisms. A unique aspect of the present invention is the ability to produce large-scale synthesis of glycoconjugates using enzymes immobilized on a solid support. This can be accomplished by providing (transfecting) an organism with a vector that includes the genes encoding the enzyme(s) of interest, and subsequently immobilizing the enzymes onto the solid support. Essentially any organism may be used to produce the enzymes in this manner as long as it can express the heterologous gene or genes. The organism may be a prokaryote or a eukaryote. Examples of prokaryotes include Esherichia coli BL21 (DE3), Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No. 49, Escherichia coli W31 10, Escherichia coli NY49, Escherichia coli KY8415, Escherichia coli NM522, Bacillus subtilis, Bacillus brevis, Bacillus amyloliquefaciens, Brevibacterium immariophilum ATCC 14068, Brevibacterium saccharolyticum ATCC 14066, Brevibacterium flavum ATCC 14067, Brevibacterium lactofermentum ATCC 13869, Corynebacterium ammoniagenes ATCC 21170, Corynebacterium glutamicus ATCC 13032, Corynebacterium acetoacidophilum ATCC 13870, Microbacterium ammoniaphilum ATCC 15354, Pseudomonas putida, and Serratia marcescens.

The eukaryote may be a yeast, an insect cell, or an animal cell. Examples of yeast include Saccharomyces cerevisiae, Saccharomyces pombe, Candida utilis, Candida parapsilosis, Candida krusei, Candida versatilils, Candida lipolytica, Candida zeylanoides, Candida guilliermondii, Candida albicans, Candida humicola, Pichia farinosa, Pichia ohmeri, Torulopsis candida, Torulopsis sphaerica, Torulopsis xylinus, Torulopsis famata, Torulopsis versatilis, Debaryomyces subglobosus, Debaryomyces cantarellii, Debaryomyces globosus, Debaryomyces hansenii, Debaryomyces japonicus, Zygosaccharomyces rouxii, Zygozaccharomyces bailii, Kluyveromyces lactis, Kluyveromyces marxianus, Hansenula anomala, Hansenula jadinii, Brettanomyces lambicus, Brettanomyces anomalus, Schizosaccharomyces pombe, Trichosporon pullulans, and Schwanniomyces alluvius.

Examples of insect cells include SF9 and SF21.

Examples of animal cells include CHO cells, BHK21, NIH 3T3, 293, and COS cells.

In a preferred embodiment, the host cell is E. coli, particularly strain DH5α, NM522 or BL21 (DE3). These organisms are well studied and amenable to recombinant technology. Use of this organism in large scale synthesis of compounds is well known in the art. Furthermore, because DH5α and NM522 are LacZ, these strains are particularly useful in methods in which selection methods need to be utilized.

The inventors also recognize that organisms that naturally express one or more enzymes, or have been engineered to express one or more enzymes, required for a particular glycoconjugate synthesis scheme may be useful. Examples include Escherichia coli which expresses the ceramide glucosyltransferase gene derived from human melanoma cell line SK-Mel-28 (Proc. Natl. Acad. Sci. USA, 1996, 93:4638), human melanoma cell line WM266-4 which produces beta 1,3-galactosyltransferase (ATCC CRL 1676), recombinant cell line such as namalwa cell line KJM-1 or the like which contains the beta 1,3-galactosyltransferase gene derived from the human melanoma cell line WM266-4 (Japanese Published Unexamined Patent Application No. 181759/94), Escherichia coli (EMBO J., 1990, 9, 3171) or Saccharomyces cerevisiae (Biochem, Biophys. Res. Commun., 1994, 201, 160) which expresses the beta 1,4-galactosyltransferase gene derived from human HeLa cells, COS-7 cell line (ATCC CRL 1651) which expresses the rat beta 1,6-N-acetylglucosaminyltransferase gene (J. Biol. Chem., 1993, 268: 15381), Sf9 cell line which expresses human N-acetylglucosaminyltransferase gene (J. Biochem., 1995, 118: 568), Escherichia coli which expresses human glucuronosyltransferase (Biochem. Biophys. Res. Commun., 1993, 196: 473), namalwa cell line which expresses human alpha 1,3-fucosyltransferase (J. Biol. Chem., 1994, 269: 14730), COS-1 cell line which expresses human alpha 1,3/1,4-fucosyltransferase (Genes Dev., 1990, 4: 1288), COS-1 cell line which expresses human alpha 1,2-fucosyltransferase (Proc. Natl. Acad. Sci. USA., 1990, 87: 6674), COS-7 cell line which expresses chicken alpha 2,6-sialyltransferase (Eur. J. Biochem., 1994, 219: 375), COS cell line which expresses human alpha 2,8-sialyltransferase (Proc. Natl. Acad. Sci. USA., 1994, 91: 7952), Escherichia coli which expresses beta 1,3-N-acetylglucosaminyltransferase, beta 1,4-galactosyltransferase, beta 1,3-N-acetylgalactosaminyltransferase or alpha 1,4-galactosyltransferase derived from Neisseria (WO 96/10086), Escherichia coli which expresses Neisseria-derived alpha 2,3-sialyltransferase (J. Biol. Chem., 1996, 271: 28271), Escherichia coli which expresses Heilcobacter pylori-derived alpha 1,3-fucosyltransferase (J. Biol. Chem., 1997, 272: 21349 and 21357), and Escherichia coli which expresses yeast-derived alpha 1,2-mannosyltransferase (J. Org. Chem., 1993, 58: 3985). Such organism when further complemented with additional sugar-nucleotide regenerating enzymes will be useful in the methods of the present invention.

### Glycoconjugates

In light of the present disclosure, it will become apparent to those of ordinary skill in the art that a great number of different glycoconjugates may be produced by the methods of the present invention if the correct enzymes, precursors, and acceptor molecules are provided to the organism.

Essentially any material may be used as a precursor or acceptor as long as it can be used as a substrate of the glycosyltransferase. The precursor and/or acceptor may be natural or synthetic. Examples include monosaccharides, oligosaccharides, monosaccharides or oligosaccharides linked to a carrier, proteins, peptides, glycoproteins, lipids, glycolipids, glycopeptides, and steroid compounds. When the glycoconjugate is a glycolipid or a glycoprotein, the glycoconjugate may be O-linked or N-linked.

Specific examples include glucose, galactose, mannose, sialic acid, N-acetylglucosamine, N-acetylgalactosamine, lactose, N-acetyllactosamine, lacto-N-biose, GlcNAc beta 1-3Gal beta 1-4Glc, GlcNAc beta 1-4Gal beta 1-4Glc, globotriose, Gal alpha 1-4Gal beta 1-4GlcNAc, 2′-fucosyllactose, 3-fucosyllactose, 3′-sialyllactose, 6′-sialyllactose, 3′-sialyl-N-acetyllactosamine, 6′-sialyl-N-acetyllactosamine, sialyllacto-N-biose, H antigen, Lewis X, Lewis A, lacto-N-tetraose, lacto-N-neotetraose, lactodifucotetraose, 3′-sialyl-3-fucosyllactose, sialyl-Lewis X, sialyl-Lewis A, lacto-N-fucopentaose I, lacto-N-fucopentaose II, lacto-N-fucopentaose III, lacto-N-fucopentaose V, LS-tetrasaccharide a, LS-tetrasaccharide b, LS-tetrasaccharide c, ( alpha 2,3) sialyllacto-N-neotetraose and derivatives thereof, serine, threonine, asparagine and peptides containing these amino acids and derivatives thereof, ceramide and derivatives thereof, saponin and derivatives thereof, and the like. The complex carbohydrate precursor can be used at a concentration of from 1 μM to 10 M. Preferably the lower range is 1 mM or 10 mM and the upper range 100 mM or 500 mM.

Examples of the glycoconjugates that may be produced by the methods of the present invention include glycoconjugates containing at least one sugar selected from glucose, galactose, N-acetylglucosamine, N-acetylgalactosamine, glucuronic acid, mannose, N-acetylmannosamine, fucose, sialic acid, lactose, N-acetyllactosamine, lacto-N-biose, GlcNAc beta 1-3Gal beta 1-4Glc, GlcNAc beta 1-4Gal beta 1-4Glc, globotriose, Gal alpha 1-4Gal beta 1-4GlcNAc, 2′-fucosyllactose, 3-fucosyllactose, 3′-sialyllactose, 6′-sialyllactose, 3′-sialyl-N-acetyllactosamine, 6′-sialyl-N-acetyllactosamine, sialyllacto-N-biose, A antigen, B antigen, Lewis X, Lewis A, lacto-N-tetraose, lacto-N-neotetraose, lactodifucotetraose, 3′-sialyl-3-fucosyllactose, sialyl-Lewis X, sialyl-Lewis A, lacto-N-fucopentaose I, lacto-N-fucopentaose II, lacto-N-fucopentaose III, lacto-N-fucopentaose V, LS-tetrasaccharide a, LS-tetrasaccharide b, LS-tetrasaccharide c, ( alpha 2,3)sialyllacto-N-neotetraose, lacto-N-difucohexaose I, lacto-N-difucohexaose II , lacto-N-hexaose, lacto-N-neohexaose, disialyllacto-N-tetraose and derivatives thereof; lipopolysaccharide (LPS), such as the LPS of Neisseria meningitidis and Neisseria gonorrhoeae, and complex carbohydrates which contain the just described complex carbohydrates. Specifically, they include complex carbohydrates which contain a sugar having a bond selected from Gal beta 1-3Glc, Gal beta 1-4Glc, Gal beta 1-3GlcNAc, Gal beta 1-4GlcNAc, Gal beta 1-3Gal, Gal beta 1-4Gal, Gal beta 1-3GalNAc, Gal beta 1-4GalNAc, Gal alpha 1-3Glc, Gal alpha 1-4Glc, Gal alpha 1-3GlcNAc, Gal alpha 1-4GlcNAc, Gal alpha 1-3Gal, Gal alpha 1-4Gal, Gal alpha 1-3GalNAc, Gal alpha 1-4GalNAc, GlcNAc beta 1-3Gal, GlcNAc beta 1-4Gal, GlcNAc beta 1-6Gal, GlcNAc beta 1-3Glc, GlcNAc beta 1-4Glc, GlcNAc beta 1-3GlcNAc, GlcNAc beta 1-4GlcNAc, GlcNAc beta 1-6GalNAc, GlcNAc beta 1-2Man, GlcNAc beta 1-4Man, GlcNAc beta 1-6Man, GalNAc beta 1-3Gal, GalNAc beta 1-4Gal, GalNAc beta 1-4GlcNAc, GalNAc alpha 1-3GalNAc, Man beta 1-4GlcNAc, Man alpha 1-6Man, Man alpha 1-3Man, Man alpha 1-2Man, GlcUA beta 1-4GlcN, GlcUA beta 1-3Gal, GlcUA beta 1-3GlcNAc, GlcUA beta 1-3GalNAc, NeuAc alpha 2-3Gal, NeuAc alpha 2-6Gal, NeuAc alpha 2-3GlcNAc, NeuAc alpha 2-6GlcNAc, NeuAc alpha 2-3GalNAc, NeuAc alpha 2-6GalNAc, NeuAc alpha 2-8NeuAc, Fuc alpha 1-3Glc, Fuc alpha 1-4Glc, Fuc alpha 1-3GlcNAc, Puc alpha 1-4GlcNAc, Fuc alpha 1-2Gal and Fuc alpha 1-6GlcNAc; and complex carbohydrates which contain the just described complex carbohydrates. In this case, the number of sugars contained in the complex carbohydrate containing the sugars may be 10⁴ or below, or 10³ or below.

### Methods of Producing Glycoconjugates

The present invention provides for the production of a large variety of oligosachharides. Generally, the method involves immobilizing at least one sugar-nucleotide regenerating or producing enzyme on at least one nickel-NTA bead; providing acceptable bioenergetic, precursor, at least one glycosyltransferase, and acceptor molecules to the beads in a reaction mixture; incubating the bead with the reaction mixture under conditions appropriate for the enzymes to produce the glycoconjugate; and recovering the glycoconjugate from the reaction mixture. As an alternative to providing the at least one glycosyltransferase to the beads, the at least one glycosyltransferase can be co-immobilized on the beads with the at least one sugar-nucleotide regenerating enzymes. Following production of the glycoconjugate, the beads can be easily separated from the reaction mixture for reuse, either with the current enzymes or following removal of the existing enzymes and immobilization of new enzymes.

Prior to immobilizing the enzymes on the beads, it may be necessary to construct a genetic vector capable of expressing the enzyme or enzymes in a quantity and configuration suitable for immobilization. For the individual cloning of a known or previously unknown gene encoding an enzyme to be used in the compositions and methods of the present invention, the coding region is isolated, through PCR or essentially any other method of isolating a nucleic acid segment, and cloned into an expression vector. A preferred expression vector is pET from Novagen. The pET vector allows for the addition of a N-terminal 6-histidine tag to the protein and a ribosomal binding site to the transcript encoding the protein. The plasmid is then transformed into a host (e.g., E-coli BL21 (DE3)) and the protein is expressed. The recombinant protein can be purified using a nickel-NTA column and characterized by an enzyme activity assay (see Example 1). Also, the histidine tag allows for immobilization onto beads containing nickel, e.g., nickel-NTA beads.

After it has been determined that the gene encodes a protein with the desired property, in preferred embodiments, the isolated gene, along with the His tag and ribosomal-binding site encoded in pET is then subcloned into pLDR20. If necessary, other proteins necessary for sugar-nucleotide regeneration and the glycosyltransferase are cloned into the same vector such that they are co-transcribed. Then, a cell lysate can be obtained from the recombinant that contains the enzyme(s) of interest. The lysate can then be used directly to immobilize the enzyme(s) onto the nickel-NTA beads. Alternatively, the enzyme(s) can be purified or semi-purified from the lysate, characterized for enzymatic activity, and later immobilized onto the beads. Due to the presence of the poly histidine tag, however, such a purification step is not necessary.

The beads having the immobilized enzymes are then used to produce the glycoconjugate. The beads are provided with an appropriate bioenergetic, along with a substrate and acceptor for the glycosyltransferase. Additionally, if the glycosyltransferase is not co-immobilized on the beads, the glycosyltransferase is provided to the beads in the reaction mixture, or by another population of beads having the glycosyltransfoase immobilized on the beads. Alternatively, the substrate or acceptor for the glycosyltransferase may be naturally produced by the enzymes involved in the biosynthetic pathway of a microorganism supplied to the bead or may comprise a molecule that has been produced using a heterologous enzyme provided to the beads. For example, FIG. 1 diagrams a method for producing Galα1,3Lac. PEP is provided to the beads as a bioenergetic; lactose is provided as an acceptor; and galactose is provided, which is eventually converted to UDP-Gal, a donor substrate for the glycosyltransferase.

The glycoconjugate product can be isolated from the reaction mixture following production using known methods. For example, after production of the glycoconjugate, the reaction mixture can be run through an ion exchange column. Essentially any resin can be utilized, but DOWEX 1×8 and DOWEX 50×8 are preferred. The eluent is then concentrated by evaporation, and the concentrated eluent is then run over a gel filtration column. For this step, Sephadex G-15 is preferred for purifying di- and trisaccharides; G-25 Sephadex is preferred for tetra- and pentasaccharides. Fractions are collected, analyzed by for the presence of the glycoconjugate by methods known in the art, such as thin layer chromatography. The fractions containing the product are pooled. Lastly, the pooled, glycoconjugate-containing fractions can be lyophilized. After lyophilization, the purified product can be characterized by nuclear magnetic resonance (NMR) and mass spectrometry.

The present invention provides methods of producing glycoconjugates containing glucose. A preferred pathway for producing glucose-containing glycoconjugates is diagrammed in FIG. 6. Also shown in FIG. 6 is a preferred vector for the production of enzymes necessary for the production of glucose-containing glycoconjugates. In this pathway, polyphosphate is provided as a bioenergetic to a bead having the enzymes of the vector immobilized thereon. Also, glucose is provided, which is converted to UDP-Glc and subsequently added to the acceptor molecule (ROH) to produce GlcOR.

In other embodiments, methods of producing glycoconjugates containing galactose are provided. A preferred pathway for producing galactose-containing glycoconjugates is diagrammed in FIG. 1. Shown in FIG. 2 is a preferred vector for the production of enzymes necessary for the production of galactose-containing glycoconjugates. In this pathway, PEP is provided as a bioenergetic to a bead having the enzymes of the vector immobilized thereon. Also, galactose is provided, which is converted to UDP-Gal and subsequently added to the acceptor molecule (lactose) to produce GIcOR.

Also provided are methods of producing glycoconjugates containing N-acetylglucosamine. A preferred pathway for producing N-acetylglucosamine-containing glycoconjugates is diagrammed in FIG. 7. Also shown in FIG. 7 is a preferred vector for the production of enzymes necessary for the production of N-acetylglucosamine-containing glycoconjugates. In this pathway, polyphosphate is provided as a bioenergetic to a bead having the enzymes of the vector immobilized thereon. Also, N-acetylglucosamine is provided, which is converted to UDP-GlcNAc and subsequently added to the acceptor molecule (ROH) to produce GlcNAcOR.

Further provided are methods of producing glycoconjugates containing N-acetylgalactosamine. A preferred pathway for producing N-acetylgalactosamine-containing glycoconjugates is diagrammed in FIG. 8. Also shown in FIG. 8 is a preferred vector for the production of enzymes necessary for the production of N-acetylgalactosamine-containing glycoconjugates. In this pathway, polyphosphate is provided as a bioenergetic to a bead having the enzymes of the vector immobilized thereon. Also, N-acetylgalactosamine is provided, which is converted to UDP-GalNAc and subsequently added to the acceptor molecule (ROH) to produce GlcNAcOR.

The present invention also provides methods of producing glycoconjugates containing glucuronic acid. A preferred pathway for producing glucuranate conjugates is diagrammed in FIG. 9. Also shown in FIG. 9 is a preferred vector for the production of enzymes necessary for the production of glucoranate conjugates. In this pathway, polyphosphate is provided as a bioenergetic to a bead having the enzymes of the vector immobilized thereon. Also, glucose is provided, which is converted to UDP-GlcA and subsequently added to the acceptor molecule (ROH) to produce GlcNAcOR by a UDP-glucuronosyltransferase (UGT).

UDP-glucuronosyltransferases (UGTs) are an abundant group of enzymes involved in de-toxification pathways for lipophilic molecules such as phenols, flavones, steroids, bile acids as well as many xenobiotics. In order to synthesize a wide variety of glucuronic acid conjugates, a UDP-GlcA transferase with liberal acceptor specificity is preferred. The significance behind the synthesis of glucuronic acid conjugates is that glucuronidation is not only involved in the detoxification of lipophilic molecules but can also enhance biological activity of a large amount of existing drugs (e.g., morphine-6-O-glucuronide is 50 times more active than morphine. In preferred embodiments, human UDP-GlcA transferase UGT2137 (EC 2.4.1.17) is used. This enzyme has an extremely broad range of substrates. UGT2137 belongs to the 2B subclass of a super-family responsible for glucuronidation of a variety of lipophilic compounds. Its acceptor Km values range from low micromolar to low millimolar. Interestingly, the Km value for UDP-GlcA donor seems dependent on the acceptor. UGT2137 is most active between pH 6.0 and 8.0. Recombinant expression of a human UDP-GlcA transferase in E. coli has been accomplished (Pillot, T. et al., Biochem. Biophys. Res. Commun. 1993, 196: 473-479).

Methods for producing hyaluronan are also provided. Hyaluronan (or hyaluronic acid; HA), a co-polymer of glucuronic acid and N-acetyiglucosamine is common in the extracellular spaces of multicellular organisms where it forms a viscous, compression resistant matrix. In prokaryotes, hyaluronate is found in the anti-phagocytic capsule formed by virulent species such as Streptococcus pyogenes and Streptococcus pneumoniae, where it helps the bacterium evade the host immune system.

Hyaluronan synthases (HAS) is the first sugar transferase shown to have the ability to utilize two different UDP-sugar donors. HAS enzymes are membrane proteins that require divalent metal ion (Mg²⁺ or Mn²⁺) for optimal activity, and show two- to five-fold higher apparent affinity for the UDP-GlcA substrate than for UDP-GlcNAc. For hyaluronic acid synthesis, it is preferred to use the Hyaluronan synthase (spHAS) from Streptococcus pyogenes, which is encoded by the spHas gene. This gene encodes a 45 kDa protein with 395 residues and was first cloned and identified in 1993 and that was later shown to be expressed in the membrane fraction (DeAngelis, P. L. et al., J. Biol. Chem. 1993, 268: 19181-19184; Tlapak-Simmons, V. L. et al., J. Biol Chem. 1999, 274: 4239-4245). spHAS activity is dependent on lipids. Maximal activity is obtained in the presence of bovine cardiolipin being about twice the activity when E. coli cardiolipin is used. The enzyme exhibits Kₘ values of 40±4 μM for UDP-GlcA and 149±3 μM for UDP-GlcNAc.

To produce large-scale synthesis of HA inexpensively, a preferred method regenerates both UDP-GlcNAc and UDP-GlcA, as well as comprises a hyaluronan sythase. This may be accomplished by co-immobilizing all of the necessary enzymes onto at least one nickel-NTA bead. The enzymes can be obtained by engineering an organism to over-express all the enzymes necessary for the precursor generation by means of a dual plasmid system (FIG. 13) comprising plasmid pLGNAP(T⁻) and plasmid pLGAP-HAS.

In preferred embodiments, these two plasmids are constructed with compatible origins of replication to be able to coexist in the same organism. They also contain different antibiotic resistance genes for easy selection of recombinant strains containing both plasmids. Both plasmids contain the X promoter region that is under the control of the temperature sensitive λcI857 repressor. This enables simultaneous expression of proteins from both plasmids once the incubation temperature is raised.

Omitting the gene of GlcNAc transferase, pLGNAP(T⁻) encodes all of the enzymes for the generation of UDP-GlcNAc (FIG. 7).

Plasmid pLGAP-HAS contains enzymes for the production of UDP-GlcA as well as the hyaluronan synthase. Since ppK gene is incorporated in the plasmid pLGNAP(T⁻), no additional copy of ppK is included in pLGAP-HAS for the synthesis of hyaluronan. This plasmid has a kanamycin resistance gene and p15A replication origin compatible for the pMB1 origin in plasmid pLGNAP(T⁻).

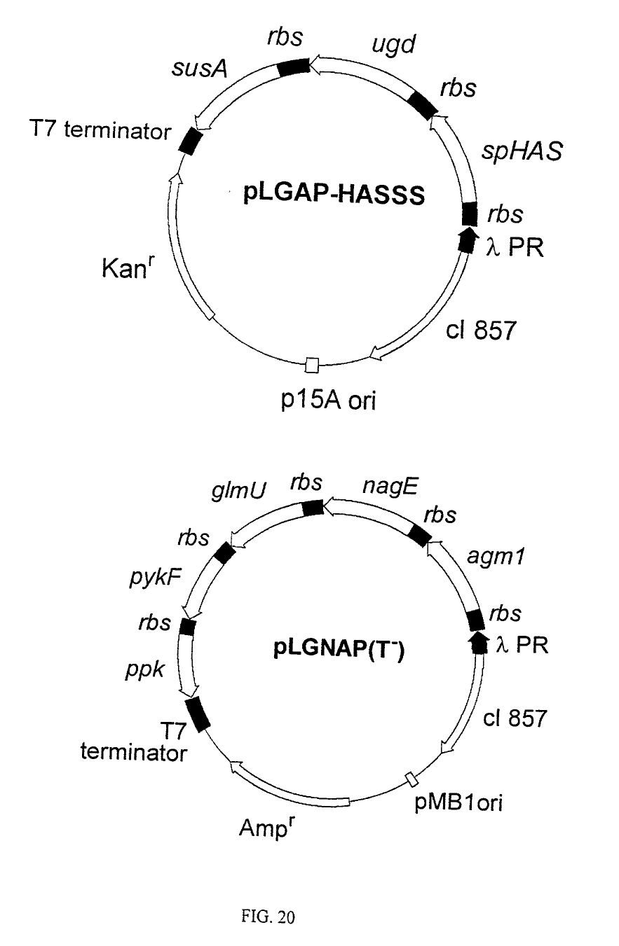

Plasmids useful in methods of synthesis of hyaluronan wherein sucrose is used are shown in FIG. 20.

Other important glycoconjugates that may be produced by the methods of the present invention are sialic acid-containing glycoconjugates. Sialic acids (N-acetylneuraminic acid, NeuNAc) exist as the terminal saccharides in a variety of glycoproteins and glycolipids on the mammalian cell surface as well as on some neuroinvasive bacteria such as Neisseria meningitis B and E. coli K1. Sialic acids containing structures play important roles in cell-cell recognition. Therefore, the synthesis of sialylated conjugates is of great importance in developing novel carbohydrate-based therapeutic agents (Fryer and Hockfield, Curr. Opin. Neurobiol. 1996, 6: 113-118; Rougon, G. Eur. J. Cell Biol. 1993, 61: 197-207.; Phillips, G. R. et al., Brain Res. Dev. Brain Res. 1997, 102: 143-155; Liu, T. Y. et al.,; J. Biol. Chem. 1971, 246: 4703-4712; Egan, W. et al., Biochemistry 1977, 16 3687-3692).

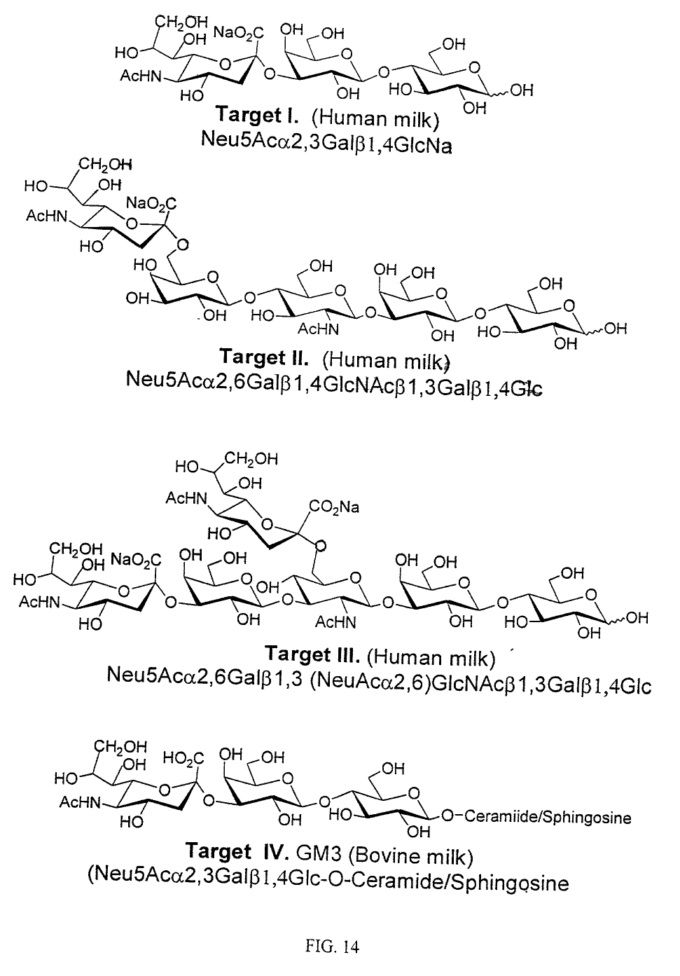

The biosynthesis of sialylated glycoconjugate typically requires CMP-NeuNAc synthesized from CTP and sialic acid. In a preferred embodiment, sialic acid aldolase (NanA), CMP-NeuNAc synthetase (NeuA), CMP kinase (Cmk), and polyphosphate kinase (Ppk) from E. coli, along with α2,3- or α2,6-sialyltransferase, SiaT, are all co-immobilized onto nickel-NTA beads. The necessary enzymes for this embodiment can be obtained by cloning all necessary genes into one plasmid (FIG. 10). Four exemplary glycoconjugates that may be produced by the methods of the present invention are shown in FIG. 14.

Also provided by the present invention are methods of producing mannose-containing glycoconjugates. A preferred pathway for producing mannose-containing glycoconjugates is diagrammed in FIG. 11. Also shown in FIG. 11 is a preferred vector for the production of enzymes necessary for the production of mannose-containing glycoconjugates (pL-ManA1A2). In this pathway, polyphosphate is provided as a bioenergetic to a bead having the enzymes of the vector immobilized thereon. Also, mannose is provided, which is converted to GDP-Man and subsequently added to the acceptor molecule (ROH) to produce ManOR.

Further provided are methods of producing fucose-containing glycoconjugates. A preferred pathway for producing fucose-containing glycoconjugates is diagrammed in FIG. 12. Also shown in FIG. 12 is a preferred vector for the production of enzymes necessary for the production of fucose-containing glycoconjugates (pL-Mfucα1,3FT). In this pathway, polyphosphate is provided as a bioenergetic to a bead having the enzymes of the vector immobilized thereon. Also, mannose is provided, which is converted to GDP-Fuc and subsequently added to the acceptor molecule (ROH) to produce FucOR.

### Conditions for Producing Glycoconjugates

The methods of the present invention are adaptable to small scale and large scale (e.g. fermentors) production of glycoconjugates. Culturing of the beads for use in the present invention may be carried out in accordance with standard laboratory incubations. One advantage of the present invention is that it utilizes a cell-free system. Consequently, unless cells are used to supply one of the necessary elements of the reactions, culturing does not require conditions and/or ingredients for supporting specific cell growth. Rather, culturing of the beads and reaction mixture can be performed under conditions appropriate for the functioning of the enzymes.

For example, the beads can be cultured in any suitably-sized reaction vessel, including tubes, flasks, beakers, and any other container suitable for holding the reaction mixture during the culturing step. Furthermore, any sort of vessel that allows a reaction mixture to flow through the vessel, such as a column or other chamber with inlet and outlet means, can also be utilized. Culturing the reaction mixture with the beads in a mixer, such as a rotor mixer or shaker, for several days at room temperature (24° C.) has been found sufficient. The use of a mixer or shaker ensures constant exposure of the beads to the components of the reaction mixture. Room temperature is generally a suitable temperature for the functioning of the various enzymes utilized by the present invention. A total reaction time of four days has been found suitable for the production of relatively large quantities of glycoconjugate involving the addition of one sugar moiety. The reaction time can be scaled up for addition of further sugar moieties.

Following the incubation, the beads can be easily separated from the reaction mixture by simple centrifugation. The speed and duration of the centrifugation step relate to the size of the beads utilized. For beads of approximately 100 rpm in diameter, centrifugation for 10 minutes at 4,000 rpm has been found acceptable for separation purposes.

Following centrifugation, the supernatant, which contains the reaction mixture and the product, can be drawn off of the pelleted beads. The glycoconjugate product can be purified from the reaction mixture by passing the mixture over a G-15 sepharose gel filtration column using water as the eluent. Glycoconjugate-containing fractions of the elute can then be pooled, lyophilized and subsequently characterized.

When a cellular system is utilized to produce the enzyme(s) for subsequent immobilization onto the beads, culturing of the organisms may be carried out in accordance with the usual culturing process.

For example, where the organism is a microorganism, such as E. coli, the medium for use in the culturing of the microorganism may be either a nutrient medium or a synthetic medium, so long as it contains carbon sources, nitrogen sources, inorganic salts and the like, which can be assimilated by the microorganism, and it can perform culturing of the microorganism efficiently.

Examples of the carbon sources include those which can be assimilated by the microorganism, such as carbohydrates (for example, glucose, fructose, sucrose, lactose, maltose, mannitol, sorbitol, molasses, starch, starch hydrolysate, etc.), organic acids (for example, pyruvic acid, lactic acid, citric acid, fumaric acid, etc.), various amino acids (for example, glutamic acid, methionine, lysine, etc.), and alcohols (for example, ethanol, propanol, glycerol, etc.). Also useful are natural organic nutrient sources, such as rice bran, cassava, bagasse, corn steep liquor, and the like.

Examples of the nitrogen sources include various inorganic and organic ammonium salts (for example, ammonia, ammonium chloride , ammonium sulfate, ammonium carbonate, ammonium acetate, ammonium phosphate, etc.), amino acids (for example, glutamic acid, glutamine, methionine, etc.), peptone, NZ amine, corn steep liquor, meat extract, yeast extract, malt extract, casein hydrolysate, soybean meal, fish meal or a hydrolysate thereof and the like.

Examples of the inorganic substances include potassium dihydrogen phosphate, dipotassium hydrogen phosphate, sodium dihydrogen phosphate, disodium hydrogen phosphate, magnesium phosphate, magnesium sulfate, magnesium chloride, sodium chloride, calcium chloride, ferrous sulfate, manganese sulfate, copper sulfate, zinc sulfate, calcium carbonate, and the like. Vitamins, amino acids, nucleic acids and the like may be added as occasion demands.

The culturing is carried out under aerobic conditions by shaking culture, aeration stirring culture or the like means. The culturing temperature is preferably from 15 to 45 ° C., and the culturing time is generally from 5 to 96 hours. The pH of the medium is maintained at 3.0 to 9.0 during the culturing. Adjustment of the medium pH may be carried out using an inorganic or organic acid, an alkali solution, urea, calcium carbonate, ammonia and the like. Also, antibiotics (for example, ampicillin, tetracycline, etc. ) may be added to the medium during the culturing as occasion demands.

In some embodiments, a microorganism transformed with an expression vector in which an inducible promoter is used. Culturing may be adjusted such that induction of the promoter is regulated (e.g., adjustment of culturing temperature). Alternatively, where a promoter is induced by a compound, an inducer may be added to the medium as occasion demands. For example, isopropyl-α-D-thiogalactopyranoside (IPTG) or the like may be added to the medium when a microorganism transformed with an expression vector containing lac promoter or lac T7 promoter is cultured, or indoleacrylic acid (IAA) or the like may by added when a microorganism transformed with an expression vector containing trp promoter is cultured.

When animal cells are used for producing the complex carbohydrate of the present invention, the preferred culture medium is generally RPMI 1640 medium, Eagle's MEM medium or a medium thereof modified by further adding fetal calf serum, and the like. The culturing is carried out under certain conditions, for example, in the presence of 5% CO₂. The culturing is carried out at a temperature of preferably from 20 to 40 ° C. for a period of generally from 3 to 14 days. As occasion demands, antibiotics may be added to the medium.

When insect cells are used for producing glycoconjugates of the present invention, culturing of the insect cells can be carried out in accordance with known processes (e.g., J. Biol. Chem., 268: 12609 (1993)).

### Kits

Further provided for by the present invention are kits containing one or more compositions of the present invention for the production of glycoconjugates. The kit may comprise nickel-NTA beads with at least one nucleotide-regenerating enzyme immobilized thereon. A kit of the present invention may comprise nickel-NTA beads with at least one nucleotide-regenerating enzyme and at least one glycosyltransferase immobilized thereon. The kit may include nickel-NTA beads without any enzymes immobilized thereon. The kit of the present invention may include various sources of the sugar-nucleotide regenerating enzymes and glycosyltransferases, including purified enzymes, semi-purified enzymes, cell lysates containing the enzymes, genetically-engineered microorganisms capable of expressing the enzymes, or natural microorganisms capable of expressing the enzymes. The kit may include a plasmid encoding at least one nucleotide-regenerating enzyme. A kit of the present invention may comprise a plasmid encoding at least one nucleotide regenerating enzyme and at least one glycosyltransferase. The kit may contain a single plasmid encoding all necessary enzymes for the production of a particular glycoconjugate. The kit may contain multiple plasmids encoding all necessary enzymes for the production of a particular glycoconjugate. A kit of the present invention may comprise an organism, which may have been transfected with a plasmid of the present invention, or a plasmid of the present invention may be included in the kit with the organism. Furthermore, the bioenergetic that the enzymes require and/or that the organism has been engineered to utilize to produce an glycoconjugate also may be included in the kit.

### EXAMPLES

The following examples are included to demonstrate embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain like or similar results without departing from the spirit and scope of the invention.

### Example 1 - Production of an Engineered Solid Support Having Immobilized GalK, GalT, GalU, and PykF

The inventors prepared beads in accordance with the present invention for UDP-Gal regeneration. The regeneration of UDP-Gal from UDP requires four enzymes: GalK, GalT, GalU and PykF. To prepare the enzymes, galK, galT, galU, and pykF genes were individually amplified from E. coli genome by polymerase chain reaction (PCR), using standard procedures. The amplified genes were inserted into the pET15b vector with a sequence coding for a N-terminal histidine tag having six histidine residues. The enzymes were expressed in E. coli BL21(DE3) with isopropyl-1-thio-β-D-galactopyranoside (IPTG) induction. Cell lysates were obtained and assayed for enzyme activity according to the following methods:

### Enzymatic Activity Assay for GalK

The activity assays for GalK were performed at room temperature (24° C.) for 30 min in a final volume of 100 μl in HEPES buffer (100 mM, pH 7.4) containing α-D-[6-³H]galactose (0.5 mM, final specific activity of 1000 cpm/nmole) and ATP (50 mM). ATP was omitted for the blank. The reaction was stopped by adding 0.8 ml of Dowex 1×8 -200 chloride anion exchange resin suspended in water [resin:H₂O (vol/vol)=1:1]. After centrifugation, supernatant (0.4 ml) was collected in a 20 ml plastic vial and ScintiVerse BD (5 ml) was added. The vial was vortexed thoroughly before the radioactivity of the mixture was counted in a liquid scintillation counter (Beckmann LS-3801 counter). One unit of enzyme activity is defined as the amount of enzyme that produces 1 μmole of galactose-1-phosphate per minute at 24° C.

### Enzymatic Activity Assay for GalT

This was a two-step assay. In the first step, GalT catalyzed reactions were performed at room temperature (24° C.) for 15 min in a final volume of 250 μl HEPES buffer (100 mM, pH 7.4) containing 1.6 mM Gal-1-P, 2.8 mM UDP-glucose, and 100 μl of enzyme solution. A blank was performed with water replacing Gal-1-P. The reaction was stopped by adding cold NaCl solution (0.5 ml, 0.15 M) and immediately transferring the tube to a boiling water bath for 5 min to terminate the reaction. The contents of the tubes were cooled to room temperature and vortexed vigorously to break up the coagulum. After centrifugation at 1400×g for 15 min, the clear supernatant (0.2 ml) was subjected to the UDP-glucose assay in a cuvette with a total volume of 1 ml containing 0.03 M Tris-acetate buffer, pH 8.7, 1.36 mM NAD, 0.2 ml sample (supernatant from the previous procedure) and 3.2 mU UDP-glucose dehydrogenase. The OD change at 340 nm was monitored by a UV spectrophotometer (HP 8453 Spectrophotometer, Hewlett-Packard Corn.). One unit of enzyme activity was defined as the amount of enzyme that produces 1 μmole of UDP-galactose per minute at 24° C.

### Enzymatic Activity Assay for GalU

A two-step assay was carried out to detect the GalU activity. In the first step, GalU catalyzed reactions were performed at room temperature (24° C.) for 15 min in a final volume of 250 μl containing 1.6 mM Glc-1-P, 2.8 mM UTP, 10 mM MgCl₂ and 100 μl enzyme solution. A blank was performed with water replacing Glc-1-P. The reaction was stopped by adding cold NaCl solution (0.5 ml, 0.15 M) and immediately transferring the tube to a boiling water bath for 5 min to terminate the reaction. The contents of the tubes were cooled to room temperature and vortexed vigorously to break up the coagulum. After centrifuge at 1400×g for 15 min, the clear supernatant (0.2 ml) was subjected to the UDP-glucose assay in a cuvette with a total volume of 1 ml containing 0.03 M Tris-acetate buffer, pH 8.7, 1.36 mM NAD, 0.2 ml sample (supernatant from the previous procedure) and 3.2 mU UDP-glucose dehydrogenase. The OD change at 340 nm was monitored by a UV spectrophotometer (HP 8453 Spectrophotometer, Hewlett-Packard Corn.). One unit of enzyme activity was defined as the amount of enzyme that produces 1 μmole of UDP-glucose per minute at 24° C.

### Enzymatic Activity Assay for Pyruvate Kinase

In a 10 mm light path cuvette was pipette successively with a total volume of 1 ml solution containing 0.1 M Tris-HCl buffer, pH 8.0, 0.5 mM EDTA, 0.1 M KCl, 10 mM MgCl₂, 0.2 mM NADH, 1.5 mM ADP, 60 mU lactate dehydrogenase, and 5 mM PEP. A blank assay was carried out with water replacing ADP. The reactions were performed at room temperature (24° C.) and the absorbance at 340 nm was monitored by a UV-spectrophotometer. One unit of enzyme activity was defined as the amount of enzyme that produces 1 μmole of pyruvate per minute at 24° C.

After determining the activity levels of the enzymes, a cell lysate mixture was prepared such that the relative activity levels of enzymes were equal. The activities of individual enzymes in the cell lysate (25 ml lysate per 1 L cell culture) were 25 U/L, 100 U/L, 100 U/L, and 50 U/L for GalK, GalT, GalU, and PykF, respectively. One unit (U) of enzyme activity is defined as the amount of enzyme that catalyzes the production of 1 μmole product per minute at 24° C. Thus, a mixture of cell lysates having relative volumes of GalK, GalT, GalU, and PykF lysates of 4:1:1:2 was prepared. Lastly, the UDP-Gal regeneration beads were obtained by incubating 120 ml of the cell lysate mixture with 40 ml of Nickel-NTA beads (3 ml lysate mixture per ml beads) for 20 minutes and washing with a Tris-HCl (20 mM, pH 8.0) containing 0.5 M NaCl.

### Example 2 - Use of Engineered Solid Support to Produce Galα1,3Galβ1,4GlcOBn

Using UDP-Gal regeneration beads according to the present invention, the inventors achieved gram scale synthesis of Galα1,3Galβ1,4GlcOBn (Table 1, entries 1 and 2). For production of this glycoconjugate, the inventors utilized nickel-NTA beads having GalK, GalT, GalU, and PykF immobilized thereon. The beads were obtained by incubation with 120 ml of a cell lysate mixture of GalK, GalT, GalU, and PykF having a volume ratio of 4:1:1:2 based on enzyme activity levels determined using procedures described above. As indicated in Example 1, the beads were produced so that equal activity levels for each of the enzymes were present on the beads. Following immobilization of the enzymes onto the beads, the beads were incubated with cell lysate containing a truncated bovine α1,3galactosyltransferase (α1,3GalT, 40 mL, 40 U) expressed in E. coli (Chen, X., et al., Biotech. Prog. 2000, 16: 595). The beads were then washed with Tris-HCl buffer (20 m M, pH 8.0) containing 0.5 M NaCl, and subsequently added to a reaction mixture containing LacβOBn (1 g, 2.4. mmol), ATP (132 mg, 240 μmol), PEP (912 mg, 4.8 mmol), UDP (100 mg, 240 μmol), Glc-1-P (73 mg, 240 μmol), Gal 540 mg, 3 mmol), MgCl₂ (10 mM), MnCl₂ (10 mM), KCl (100 mM) in HEPES buffer (100 mM, pH 7.5) to a total volume of 250 mL. The reaction was stirred at room temperature (24° C.) for four days, when thin-layer chromatographic analysis [i-PrOH:NH₄OH:H₂O=7:3:2 (v/v/v)] indicated the reaction was complete. Following the reaction, the beads were separated from the reaction mixture by centrifugation at 4,000 rpm for 10 minutes at room temperature and washed with Tris-HCl buffer (20 mM, pH 8.0) containing 0.5 M NaCl to prepare them for later reuse. The glycoconjugate product was purified by passing the reaction mixture over a G-15 sepharose gel filtration column using water as the mobile phase. The trisaccharide-containing fractions were identified by thin layer chromatography analysis and were pooled and lyophilized to give Galα1,3Galβ1,4GlcOBn. Using this procedure, the inventors obtained 1.03 grams of product, which corresponded to a 72% yield based on acceptor LacOBn.

Following this production, the beads were subsequently used for three additional production runs during a three week period while maintaining 90% enzyme activity based on α1,3GalT.

The inventors also produced Galα1,3Galβ1,4GlcOBn using beads having the sugar-regenerating enzymes immobilized on the beads while providing the glycosyltransferase in solution to the bead (Table 1, entry 2). For this production, the above procedure was followed except that GaiT was not immobilized on the beads. Rather, the N-terminal His-tagged recombinant α1,3GalT was purified over a Ni-NTA column and treated with thrombin solution for 16 hours to cleave the His-tag from the enzyme. Then, the tag was removed from the enzyme solution by dialysis against Tris-HCl buffer (20 mM, pH 7.9) containing 10% glycerol. The enzyme solution was then added to the bead reaction mixture for the synthesis of Galα1,3Galβ1,4GlcOBn. For this production with the glycosyltransferase provided to the bead in solution, a yield of 78% was achieved.

The versatility of the UDP-Gal regeneration beads is exemplified by the following examples of syntheses of a variety of glycoconjugates (Table 1, entries 3 and 4). A combination of the beads with bovine β1,4galactosyltransferase (Sigma) in solution readily produced Galβ1,4GlcNAc with 92% yield. Galβ1,4GlcNAc is one of the most common sugar sequences existing in a variety of natural glycoconjugates. A combination of the beads with α1,4galactosyltransferase immobilized on beads produced Galα1,4Galβ1, GlcOBn with 86% yield (Table 1, entry 4). This sugar sequence (called globotriose Gb₃) is a trisaccharide portion of globotriaosylceramide, which is the receptor of E. coli derived verotoxin (VT). VT binding to the Gb₃ is believed to be a crucial step in the development of hemorrhagic colitis, and hemolytic uremic syndrome commonly known as ‘Hamburger disease’. Synthetic Gb₃ derivatives could be effective inhibitors of this interaction and have important pharmaceutical potential. The α1,4galactosyltransferase (lgtC gene) used to produce the enzyme was cloned from Neisseria meningitidis and expressed in E. coli BL21 (DE3) (Kowal and Wang, unpublished data).

Another powerful synthetic potential of the beads of the present invention is the ability to utilize some unnatural monosaccharides as starting materials to synthesize unnatural glycoconjugates. For example, when 2-deoxygalactose was used as starting monosaccharide instead of galactose, a combination of the UDP-Gal regeneration beads with α1,3GalT co-immobilized on the beads generated a novel 2-deoxy α-Gal epitope (Table 1, entry 5). Similarly, the use of 1-¹³C labeled galactose generated 1-¹³C labeled (α-Gal epitope (Table 1, entry 6).

The UDP-Gal regeneration beads can be used in combination with multiple galactosyltransferases. For example, both α1,3GalT and β1,4GalT can be simultaneously immobilized onto the beads to generate specific Galα1,3Galβ1,4Glc sequence-producing beads. Using two equivalents 1-¹³C labeled galactose and four equivalents of PEP as starting materials, double 1-¹³C labeled trisaccharide was produced (Table 1, entry 7) from GlcNAc when enough reaction time (10 days) was given. Disaccharide (1-¹³C)Galβ1,4GlcNAc was formed as an intermediate as indicated by TLC during the reaction process. Similarly, using two equivalents of galactose as starting sugar and GlcNAcβ1,3Galβ1,3GlcN₃ as an acceptor, pentasaccharide (Table 1, entry 8) was produced with 76% overall yield.

TABLE 1
Preparative syntheses of oligosaccharides with UDP-Gal regeneration superbeads.

| Entry | GalTs | Starting | Gal | Acceptor |
|---|---|---|---|---|
| 1 | α1,3GalT on bead | 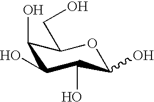 | (1 eq.) | 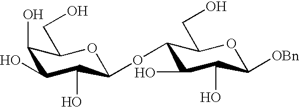 |
| 2 | α1,3GalT in solution | 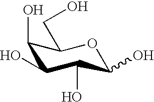 | (1 eq.) | 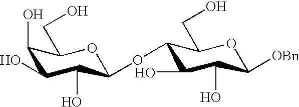 |
| 3 | β1,4GalT in solution | 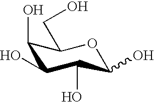 | (1 eq.) | 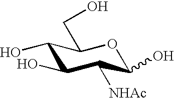 |
| 4 | α1,4GalT on bead | 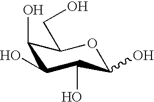 | (1 eq.) | 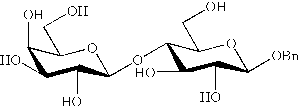 |
| 5 | α1,3GalT on bead | 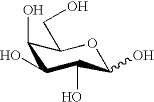 | (1 eq.) | 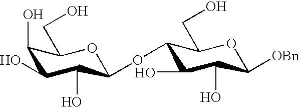 |
| 6 | α1,3GalT on bead | 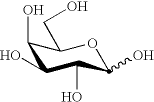 | (1 eq.) | 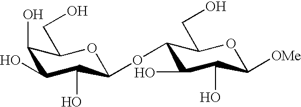 |
| 7 | α1,3GalT on bead β1,4GalT on bead | 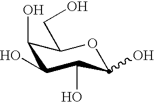 | (2 eq.) | 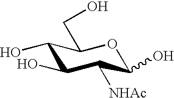 |
| 8 | α1,3GalT on bead β1,4GalT on bead | 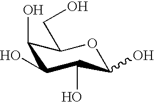 | (2 eq.) | 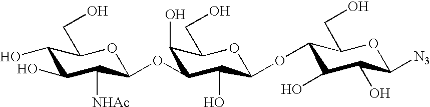 |

| Entry | Products | Yields (%) |
|---|---|---|
| 1 | 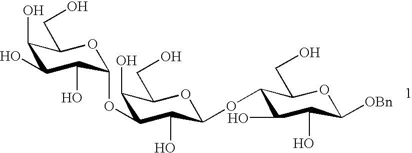 | 85 (72^{a}) |
| 2 | 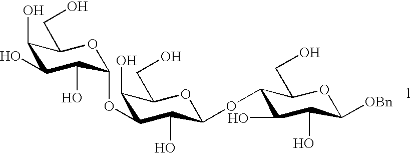 | 78 |
| 3 | 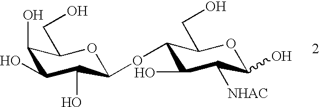 | 92 |
| 4 | 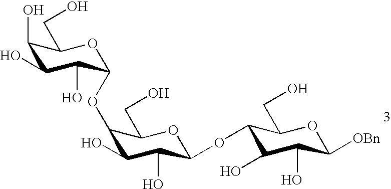 | 86 |
| 5 | 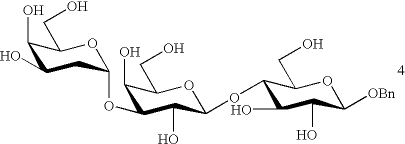 | 69 |
| 6 | 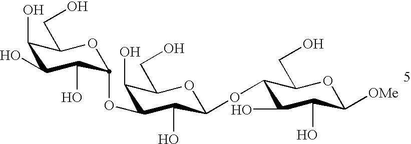 | 83 |
| 7 | 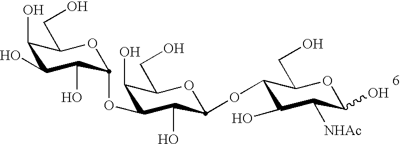 | 95 |
| 8 | 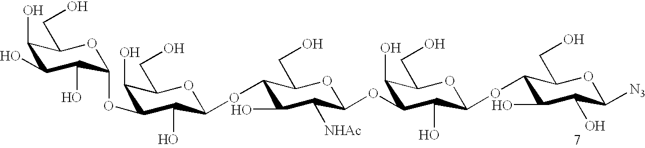 | 76 |

^{a}Gram-scale synthesis. Others are 100 mg-scale reactions.

### A. Pep

Prior to producing the beads, the inventors confirmed the biochemical scheme of FIG. 1 using purified enzymes. Each of the enzymes involved in the synthetic pathway was individually cloned and overexpressed using the pET15b vector system (Fang et al., J. Am. Chem. Soc. 1998, 120, 6635-6638; Chen et al., Biotech. Lett. 1999, 21, 1131-1135). Since each enzyme contained a hexo-histidine tag in the N-terminus, the purification was simplified by passing a cell lysate through a single Ni²⁺-NTA column.

The specific activity of the purified enzymes was determined by first determining the enzyme activity (units), next determining the amount of the enzyme present in mg using common techniques for concentration determination, such as the lowry method (J. Biol. Chem. 1951, 193:265-275). Specific activity was then determined by determining the ratio of activity: amount (units/mg.). The specific activity (U/mg) of each enzyme was as follows: GalK (2); GalT (5); GalU (10); and PykF (3).

Stepwise radioactivity assays using combinations of the purified enzymes were performed. Three steps of radioactivity assay were carried out using the combination of purified enzymes. Radio-labeled galactose was used. The first step assay was to test the combined activity of GalK, GalT and α1,3GalT. The enzyme assay was performed at 37° C. for 2 h in a final volume of 100 μl containing HEPES buffer (100 mM, pH 7.4), MnCl₂ (10 mM), D-[6-³H]galactose 90.5 mM, 20,000 dpm), ATP (5 mM), UDP-Glc (5 mM0, Lac-grease (0.14 mM), and enzyme solutions (20 μl of GalK, GalT and α1,3galT respectively). ATP was omitted for blank. The reaction was stopped by adding 0.5 ml of ice cold water.

The mixture was then pass through a Sep-Pak C₁₈ cartridge pre-washed with MeOH (20 ml) and H₂O (20 ml). The cartridge was then washed with 30 ml of water before the radio-labeled product (Galα1,3Lac-grease) was eluted with MeOH (3.5). The eluate was collected in a 20 ml plastic vial and ScintiVerse BD (10 ml) was added. The vial was vortexed thoroughly before the radioactivity of the mixture was counted in a liquid scintillation counter (Beckmann LS-3801 counter).

The second step assay was to test the combined activities of Galk, GalT, α1,3GalT and GalU. The procedures were as same as the first step assay except that the reaction mixture consisted of HEPES buffer (100 mM, pH 7.4), MnCl₂ (10 mM), D-[6-³H]galactose (0.5 mM0, ATP (5 mM0, UTP (5 mM), Glc-1 phosphate (0.5 mM), Lac-grease (0.14 mM), and enzyme solutions of GalK, GaiT, (xl,3GalT, and GalU (20μrespectively). The third step assay was the whole cycle assay, the reaction mixture consisted of HEPES buffer (10 mM, pH 7.4), MnCl₂ (10 mM), KCl (100 mM), D-[6-³H]galactose (0.5 mM), ATP (5 mM), 5 mM PEP, 0.5 mM Glc-1 phosphate, UDP (0.5 mM0, Lac-grease (0.14 mM0, and enzyme solutions of all of the five enzymes (20 μl of GalK, GalT, α1,3GalT, GalU, and PykF respectively) with a final volume of 150 μl.

The acceptor for the α1,3GalT in this assay was LacO(CH₂)₇CH₃ (Lac-grease), a lactose derivative containing a hydrophobic part that can bind to the Sep-Pak C₁₈ cartridge. According to the regeneration cycle, radio labeled *Gal was converted to radio-labeled product *Galα1,3 LacO(CH₂)₇CH₃ by stepwise combination of different recombinant enzymes along the pathway (step 1-3 in Table 2). The trisaccharide product was separated from *Gal by passing through Sep-Pak C₁₈ cartridge and eluted with methanol. The radioactivity measured by a scintillation counter presented the amount of the product formed. All of these radioactivity assays were achieved with reasonable high conversions, indicating that each individual recombinant enzyme did function as designed in the regeneration cycle.

The results are shown in Table 2.

TABLE 2
Radioactivity assays for the production of α-Gal with purified
recombinant enzymes following the biosynthetic pathway.^{a}

| | | | Product |
|---|---|---|---|
| Steps | Enzymes | Starting Material | (%) |
| 1 | GalK + GalT + α1,3GalT | ATP + Gal + Lac-grease + | 65 |
| | | UDP-Glc | |
| 2 | GalK + GalT + α1,3GalT + | ATP + Gal + Lac-grease + | 50 |
| | GalU | UTP + Glc-1-P (cat.) | |
| 3 | GalK + GalT + α1,3GalT + | ATP + Gal + Lac-grease + | 50 |
| | GalU + PykF | PEP + EDP (cat.) + | |
| | | Glc-1-P (cat.) | |
| ^{a}The acceptor for the α1,3GalT in these assays was LacO(CH₂)₇CH₃(lac-grease). It has a hydrophobic part that can bind to the Sep-Pak C₁₈ cartridge. Radio-labeled *Gal was converted to radio-labeled product *Galα1,3LacO(CH₂)₇CH₃ by the combination of recombinant enzymes. The trosaccharide product was separated from *Gal by passing through a Sep-Pak C₁₈ cartridge and eluted with methanol. The radioactivity measured by a scintillation | | | |
| #counter presented the amount of the product formed. | | | |

Finally, stepwise enzymatic synthesis of α-Gal by the use of beads was preformed as follows: The superbead (40 mL, obtained by incubation with 120 mL of cell lysate mixture of GalK, GalT, GalU and PykF with a volume ratio of 4:1:1:2) was incubated with cell lysate of α1,3GalT (40 mL, 40 U), washed by Tris-HCl buffer (20 mM, pH 8.0) containing 0.5 M of NaCl, and added to a reaction mixture of LacOBn (1.04 g, 2.4 mmol), ATP (132 mg, 240 μmol), PEP (912 mg, 4.8mmol), UDP (100 mg, 240 μmol), Glc-1-P (us mg, 240 μmol), Gal (540 mg, 3 mmol), Gal (540 mg, 3 mmol), MgCl₂ (10 mM), McCl₂ (10 mM0, KCl (100 mM) in HEPES buffer (100 mM, pH 7.5) to a total volume of 250 mL. The reaction was stirred at room temperature (24° C.) for four days. When thin-layer chromatographic analysis [I-PrOH:NH4OH:H₂O=7:3:2 (v/v/v)] indicated that reaction was complete, the superbeads were separated from the reaction mixture by centrifugation and washed for another batch of reaction. Product was purified from reaction mixture by Sephadex G-15 gel filtration column with water as the mobile phase. The trisaccaride-containing fractions were pooled and lyophilized to give Galα1.3LacOBn (1.03 gram, 72% yield based on acceptor LacOBn). The beads were then reused three times during three-week period retaining 90% enzyme activity based on α1,3GalT. After several repeated syntheses, the deactivated enzymes were removed from the nickel beads, and the beads were recharged for more uses.

### B. ATP

Compared to PEP, ATP is relatively inexpensive. Thus, the use of ATP as a bioenergetic is desirable. A protocol for using ATP as the bioenergetic in the production of Galα1,3LacOBn is as follows.

Nickel-NTA beads (40 mL, obtained by incubation with 120 mL of cell lysate mixture of GalK, GalT, GA1U and NdK with a volume ratio nof 4:1:1:2) are incubated with cell lysate of α1,3GalT (40 mL, 40 U), washed by Tris-HCl buffer (20 mM, pH 8.0) containing 0.5 M of NaCl, and added to a reaction mixture of LacOBn (1.04 g, 2.4 mmol), ATP (2.64 g, 4.8 mmol), UDP (100 mg, 240 μmol), Glc-1-P (73 mg, 240 μmol), Gal (540 mg, 3 mmol), MgCl₂ (10 mM0, MnCl₂ (10 mM0, KCl (100 mM) in HEPES buffer (100 mM, pH 7.5) to a total volume of 250 mL. The reaction is stirred at room temperature (24° C.) for four days. When thin-layer chromatographic analysis [I-PrOH:NH₄OH:H₂O=7:3:2 (v/v/v)] indicates that the reaction is complete, the superbeads are separated from the reaction mixture by centrifugation and washed for another batch of reaction. Product is purified from reaction mixture by Sephadex G-15 gel filtration column with water as the mobile phase. The trisaccharide-containing fractions are pooled and lyophilized to give Galα1,3LacOBn (1.03 grams, 72% yield based on acceptor (LacOBn). The beads can then be reused. After several repeated syntheses, the deactivated enzymes can be removed from the nickel beads, and the beads can be recharged for more uses.

### C. Polyphosphate

Even cheaper than ATP is polyphosphate. A protocol for using polyphosphate as the bioenergetic in the production of Galα1,3LacOBn is as follows:

Nickel NTA beads (40 mL, obtained by incubation with 120 mL of cell lysate mixture of GalK, GalT, GalU and PpK with a volume ratio of 4:1:1:5) are incubated with cell lysate of α1,3GalT (40 mL, 40 U), washed by Tris-HCl buffer (20 mM, pH 8.0) containing 0.5 M of NaCl, and added to a reaction mixture of LacOBn (1.04 g, 2.4 mmol), ATP (132 mg, 240 μmol), Pₙ (4.8 mmol), UDP (100 mg, 240 μmol), Glc-1-P (73 mg, 240 μmol), Gal (540 mg, 3 mmol), MgCl₂ (10 mM), KCl (100 mM) in HEPES buffer (100 mM, pH 7.5) to a total volume of 250 mL. The reaction is stirred at room temperature (24° C.) for four days. When thin-layer chromatographic analysis [I-=PrOH:NH₄OH:H₂O=7:3:2 (v/v/v)] indicates that the reaction is complete the superbeads are separated from the reaction mixture by centrifugation and washed for another batch of reaction. Product is purified from reaction mixture by Sephadex G-15 gel filtration column with water as the mobile phase. The trisaccharide-containing fractions are pooled and lyophilized to give Galα1,3LacOBn (1.03 gram, 72% yield based on acceptor LacOBn). The beads can then be reused. After several repeated syntheses, the deactivated enzymes can be removed from the nickel beads and the beads can be recharged for more uses.

### O₂

A protocol for using O₂ as the biogenetic in the production of Galα1,3LacOBn is as follows:

Nickel-NTA beads (40 mL, obtained by incubation with 240 mL of cell lysate mixture of GalK, GalT, GalU, NdK, PoxB, AcK, and Ppa) are incubated with cell lysate of α1,3GalT (40 mL, 40U), washed by Tris-HCl buffer (20 mM, pH 8.0) containing 0.5 M of NaCl, and added to a reaction mixture of LacOBn (1.04 g, 2.4 mmol), ATP (132 mg, 240 μmol), Pyruvate (4.8 mmol), UDP (100 mg, 240 μmol), Glc-1-P (73 mg, 240 μmol), Acetate phosphate (240 μmol), Gal (540 mg, 3 mmol), MgCl₂ (10 mM0, MnCl₂ (10 mM), KCl (100 mM0 in HEPES buffer (100 mM, pH 7.5) to a total volume of 250 mL. The reaction is stirred at room temperature (24° C.) for four days. When thin-layer chromatographic analysis [i-PrOH:NH₄OH:H₂O=7:3:2 (v/v/v] indicates that the reaction is complete, the superbeads are separated from the reaction mixture by centrifugation and washed for another batch of reaction. Product is purified from the reaction mixture by Sephadex G-15 gel filtration column with water as the mobile phase. The trisaccaride-containing fractions are pooled and lyophilized to give Galα1,3LacOBn (1.03 gram, 72% yield based on acceptor LacOBn). The beads can then be reused. After several repeated syntheses, the deactivated enzymes can be removed from the nickel beads, and the beads can be recharged for more uses.

### E. Sucrose Using Sucrose Synthases (ss)

A protocol for producing sucrose using sucrose synthases in the bead system is as follows:

Nickel-NTA beads (20 mL, obtained by incubation with 60 mL of cell lysate mixture of SS and GalE with a volume ratio of 1:2) are incubated with cell lysate of α1,3GalT (40 mL, 40 U), washed by Tris-HCl buffer (20 mM, pH 8.0) containing 0.5 M of NaCl, and added to a reaction mixture of LacOBn (1.30 g, 3.0 mmol), UDP (125 mg, 300 μmol), Sucrose (1.44 g, 4.2 mmol), MgCl₂ (10 mM), in MES buffer (50 mM, pH 6.0) to a total volume of 120 mL. The reaction is stirred at room temperature (24° C.) for four days. When thin-layer chromatographic analysis [i-ProOH:NH₄OH:H₂O=7:3:2(v/v/v)] indicates that the reaction is complete, the superbeads are separated from the reaction mixture by centrifugation and washed for another batch of reaction. Product is purified from the reaction mixture by Sephadex G-15 gel filtration column with water as the mobile phase. The trisaccharide-containing fractions are pooled and lyophilized to give Galα1,3LacOBn (1.03 gram, 72% yield based on acceptor LacOBn). The beads can then be reused. After several repeated syntheses, the deactivated enzymes can be removed from the nickel beads, and the beads can be recharged for more uses.

The references cited in this disclosure, except in which they may contradict any statements or definitions made herein, are incorporated by reference in their entirety.

The following table lists abbreviations used herein (Table 3).

Table 3 is complete, the superbeads are separated from the reaction mixture by centrifugation and washed for another batch of reaction. Product is purified from the reaction mixture by Sephadex G-15 gel filtration column with water as the mobile phase. The trisaccaride-containing fractions are pooled and lyophilized to give Galα1,3LacOBn (1.03 gram, 72% yield based on acceptor LacOBn). The beads can then be reused. After several repeated syntheses, the deactivated enzymes can be removed from the nickel beads, and the beads can be recharged for more uses.

### E. Sucrose Using Sucrose Synthases (ss)

A protocol for producing sucrose using sucrose synthases in the bead system is as follows:

Nickel-NTA beads (20 mL, obtained by incubation with 60 mL of cell lysate mixture of SS and GalE with a volume ratio of 1:2) are incubated with cell lysate of α1,3GalT (40 mL, 40 U), washed by Tris-HCI buffer (20 mM, pH 8.0) containing 0.5 M of NaCl, and added to a reaction mixture of LacOBn (1.30 g, 3.0 mmol), UDP (125 mg, 300 μmol), Sucrose (1.44 g, 4.2 mmol), MgCl₂ (10 mM), in MES buffer (50 mM, pH 6.0) to a total volume of 120 mL. The reaction is stirred at room temperature (24° C.) for four days. When thin-layer chromatographic analysis [i- ProOH:NH₄OH:H₂O=7:3:2(v/v/v)] indicates that the reaction is complete, the superbeads are separated from the reaction mixture by centrifugation and washed for another batch of reaction. Product is purified from the reaction mixture by Sephadex G-15 gel filtration column with water as the mobile phase. The trisaccharide-containing fractions are pooled and lyophilized to give Galα1,3LacOBn (1.03 gram, 72% yield based on acceptor LacOBn). The beads can then be reused. After several repeated syntheses, the deactivated enzymes can be removed from the nickel beads, and the beads can be recharged for more uses.

The references cited in this disclosure, except in which they may contradict any statements or definitions made herein, are incorporated by reference in their entirety.

The following table lists abbreviations used herein (Table 3).

TABLE 3

| Abbreviation | Definition |
|---|---|
| AcK | Acetate kinase |
| ADP | adenosine 5′-diphosphate |
| Alg1 | GDP-Man: Dol-PP-GlcNAc beta- |
| | mannosyltransferase |
| Alg2 | α1,3-mannosyltransferase |
| ATP | adenosine 5′-triphosphate |
| Cmk | CMP kinase |
| CMP | Cytosine 5′-monophosphate |
| CMP-NeuNAc | Cytosine 5′-monophospho-N-acetylneuraminic acid |
| cpsG(manS) | encodes PMM |
| cpsB(manC) | encodes GMP |
| CTP | Cytosine 5′-triphosphate |
| dATP | deoxyadenosine 5′-triphosphate |
| dCMP | deoxycytosine 5′-monophosphate |
| Eagle's MEM | Eagle's minimum essential medium |
| EDTA | Ethylenediaminetetraacetic acid |
| FucOR | Fucose terminated glycoconjugate |
| FucT | fucosyltransferase |
| Glk | glucose kinase |
| Gal | galactose |
| Gal-1-P | galactose-1-phosphate |
| GalE | UDP-Gal 4-Epimerase, UDP-Glc 4-Epimerase |
| GalK | galactokinase |
| GalNAc | N-acetylgalactosamine |
| GalNAc-1-P | N-acetylgalactosamine-1-phosphate |
| GalT | galactose-1-phosphate uridylyltransferase |
| GalU | glucose-1-phosphate uridylyltransferase |
| GDP-Fuc | Guanosine 5′-diphosphofucose |
| GDP-Man | Guanosine 5′-diphosphomannose |
| GFS | GDP-L-fucose synthetase |
| Glc-1-P | glucose-1-phosphate |
| GlcA | Glucuronic acid |
| GlcNAc | N-acetylglucosamine |
| GlcNAcOR | Glycoconjugate terminated with N- |
| | acetylglucosamine |
| GlcOR | Glycoconjugate terminated with glucose |
| GMD | GDP-D-mannose 4,6-dehydratase |
| GMER | GDP-4-keto-6-deoxy-D-mannose |
| | epimerase/reductase |
| GMP | GDP-mannose pyrophosphorylase |
| GST | Glutathione S-transferase |
| GTP | Guanosine 5′-triphosphate |
| HAS | hyaluronan synthases |
| HEPES | N-(2-Hydroxyethyl)piperazine-N′-(2-ethanesulfonic |
| | acid) |
| HPLC | high performance liquid chromatography |
| IAA | indoleacrylic acid |
| LgtA | β1,3GlcNAc transferase |
| IPTG | isopropyl-β-D-thiogalactopyranoside |
| IRES | internal ribosome entry site |
| ITP | Inositol-5′-triphosphate |
| Lac | lactose |
| Lac-grease | LacO(CH₂)₇CH₃ |
| LacNAc | N-acetylactosamine |
| lacZ | β-galactosidase |
| LPS | lipopolysaccharide |
| LPS O-antigen | lipopolysaccharide O antigen |
| manB | Phosphomannomutase gene |
| manC | mannose-1-phosphate guanyltransferase gene, GDP- |
| | mannose pyrophosphorylase gene |
| ManNAc | N-acetylmannosamine |
| ManOR | Glycoconjugate terminated with mannose |
| NAD | Nicotinamide adenine dinucleotide |
| NADH | Nicotinamide adenine dinucleotide (reduced form) |
| NanA | N-acetylneuraminate lyase, sialic acid aldolase |
| nana | sialic acid aldolase gene |
| NeuA | CMP-Neu NAG synthetase |
| neuA | CMP-NeuNAc synthetase |
| NeuAc | N-acetylneuraminic acid |
| NeuNAc | N-acetylneuraminic acid |
| nickel-NTA | nickel-nitrilotriacetic acid |
| NMR | nuclear magnetic resonance |
| OD | optical density |
| PEP | phospho(enol)pyruvate |
| PgM | phosphoglucomutase |
| PMI | phosphomannose isomerase |
| PMM | phosphomannomutase |
| PoxB | Pyruvate oxidase |
| ppa | Pyrophosphatase gene |
| Ppase | pyrophosphatase |
| Ppi | pyrophosphate |
| PpK | polyphosphate kinase |
| PTS | PEP-dependent transporter system |
| PykA | pyruvate kinase |
| PykF | pyruvate kinase |
| rbs | ribosomal binding site |
| RBS | ribosome binding sequence |
| rfbK | encodes PMM |
| rfbM | encodes GDP-mannose pyrophosphorylase |
| SDS-PAGE | sodium dodecyl sulfate polyacrylamide gel |
| | electrophoresis |
| SiaT | α2,3 (or α2,6)-sialyltransferase |
| spHas | Hyaluronan synthase from Streptococcus pyogenes |
| SS | sucrose synthase |
| susA | Sucorose synthetase gene |
| UDP | uridine 5′-diphosphate |
| UDP-Gal | uridine 5′-diphosphogalactose |
| UDP-GlcA | uridine 5′-diphosphoglucuronic acid |
| UDP-GalNAc | uridine 5′-diphospho-N-acetylgalactosamine |
| UDPGDH | UDP-Glc 6-dehydrogenase |
| UDP-GlcNAc | uridine 5′-diphospho-N-acetylglucosamine |
| UDP-Glc | uridine 5′-diphosphoglucose |
| UGT | UDP-glucuronosyltransferase |
| UTP | uridine 5′-triphosphate |
| α2,6SiaT | SiaT 0160 |
| α-Gal | alpha-galactose epitopes |

The foregoing disclosure is the best mode devised by the inventors for practicing the invention. It is apparent, however, that glycoconjugate synthesis systems incorporating various modifications and variations may be conceivable to one skilled in the art of glycoconjugate synthesis. Inasmuch as the foregoing disclosure is intended to enable one skilled in the pertinent art to practice the instant invention, it should not be construed to be limited thereby but should be construed to include such aforementioned obvious variations and be limited only by the spirit and scope of the following claims:

We claim:

1. An in vitro glycoconjugate-producing system comprising: a solid support;
one or more sugar nucleotide producing enzyme(s) selected from the group consisting of GalK, GalT, GalU, PykF, Ndk, PpK, AcK, PoxB, Ppa, PgM, NagE, Agml, glmU, a GalNAc kinase, a pyrophosphorylase, Ugd, NanA, Cmk, NeuA, Alg2, Alg1, SusA, GalE, GMP, GMD, and GFS; and
one or more glycosyltransferase enzyme(s) selected from the group consisting of galactosyltransferases, glucosyltransferases, N-acetylglucosaminyltransferases, N-acetylgalactosaminyltransferases, glucuronyltransferases, sialyltransferases, mannosyltransferases, and fucosyltransferases;
wherein at least one of said enzymes is immobilized on said solid support.

2. The in vitro glycoconjugate-producing system of claim 1, wherein said solid support comprises NTA-Ni²⁺ nicrospherical beads.

3. The in vitro glycoconjugate-producing system of claim 1, wherein at least one of said nucleotide producing enzymes is immobilized on said solid support.

4. The in vitro glycoconjugate-producing system of claim 1, wherein at least one of said glycosyltransferase enzymes is immobilized on said solid support.

5. The in vitro glycoconjugate-producing system of claim 1, further comprising a plasmid encoding said sugar nucleotide producing enzymes.

6. The in vitro glycoconjugate-producing system of claim 5, further comprising a cell transfected with said plasmid.

7. The in vitro glycoconjugate-producing system of claim 1, further comprising a plasmid encoding said glycosyltransferase enzyme.

8. The in vitro glycoconjugate-producing system of claim 7, further comprising a cell transfected with said plasmid.

9. The in vitro glycoconjugate-producing system of claim 1, further comprising a cell comprising heterologous genes encoding said one or more sugar nucleotide producing enzymes and said one or more glycosyltransferase.

10. The in vitro glycoconjugate-producing system of claim 1, comprising 2 or more sugar nucleotide producing enzymes.

11. The in vitro glycoconjugate-producing system of claim 1, comprising 3 or more sugar nucleotide producing enzymes.

12. The in vitro glycoconjugate-producing system of claim 1, comprising 4 or more sugar nucleotide producing enzymes.

13. The in vitro glycoconjugate-producing system of claim 1, comprising GalK, GalT, and GalU.

14. The in vitro glycoconjugate-producing system of claim 13 further comprising PykF.

15. The in vitro glycoconjugate-producing system of claim 13 further comprising Ndk.

16. The in vitro glycoconjugate-producing system of claim 13 further comprising Ppk.

17. The in vitro glycoconjugate-producing system of claim 13 further comprising PoxB, Ndk and Ppa.

18. The in vitro glycoconjugate-producing system of claim 1, comprising SusA and GalE.

19. The in vitro glycoconjugate-producing system of claim 1, wherein said solid support comprises a bead of material selected from the group consisting of agarose, methacrylate, cellulose, polystyrene, polystyrene coated ferric oxide, silica coated ferric oxide, and nitriloacetic acid.

20. The in vitro glycoconjugate-producing system of claim 1, wherein said solid support is attached to one member of a binding pair.

21. The in vitro glycoconjugate-producing system of claim 20, wherein said one member of a binding pair is selected from the group consisting of Ni²⁺, glutathione, monoclonal antibodies, polyclonal antibodies, Protein A, Protein G, and avidin.

22. The in vitro glycoconjugate-producing system of claim 20, wherein said one or more sugar nucleotide producing enzymes is attached to a second member of a binding pair.

23. The in vitro glycoconjugate-producing system of claim 22, wherein said second member of a binding pair is selected from the group consisting of poly-histidine, glutathione-S-transferase fusion protein, antigen, biotin, and solid support binding domain.

24. The in vitro glycoconjugate-producing system of claim 20, wherein said glycosyltransferase is attached to a second member of a binding pair.

25. The in vitro glycoconjugate-producing system of claim 22, wherein said second member of a binding pair is selected from the group consisting of poly-histidine, glutathione-S-transferase fusion protein, antigen, biotin, and solid support binding domains.

26. The in vitro glycoconjugate-producing system of claim 1, wherein each sugar nucleotide producing enzyme comprises a tag sequence.

27. The in vitro glycoconjugate-producing system of claim 26, wherein the tag sequence is polyhistidine.

28. The in vitro glycoconjugate-producing system of claim 1, wherein each glycosyltransferase comprises a tag sequence.

29. The in vitro glycoconjugate-producing system of claim 28, wherein the tag sequence is polyhistidine.

30. The in vitro glycoconjugate-producing system of claim 1, further comprising an epimerase.

31. The in vitro glycoconjugate-producing system of claim 30, wherein the epimerase is UDP-Gal-4-epimerase.

32. The in vitro glycoconjugate-producing system of claim 1, further comprising a fusion protein of an epimerase and at least one glycosyltransferase.

33. The in vitro glycoconjugate-producing system of claim 1, wherein the glycosyltranferase is α1,3-galactosyltransferase.

34. The in vitro glycoconjugate-producing system of claim 1, further comprising a second solid support.

35. The in vitro glycoconjugate-producing system of claim 34, wherein at least one sugar nucleotide producing enzyme is immobilized on the first solid support and at least one glycosyltransferase is immobilized on the second solid support.

36. A reaction vessel containing the in vitro glycoconjugate-producing system of claim 1.

37. The reaction vessel of claim 36, further comprising a reaction solution.

38. The reaction vessel of claim 37 in which the glycosyltransferase is in the reaction solution.

39. A method of producing a glycoconjugate comprising contacting the in vitro glycoconjugate-producing system of claim 1 with a bioenergetic, an acceptor, and a precursor to produce a glycoconjugate.

40. The method of claim 39, further comprising the step of purifying the glycoconjugate produced.

41. The method of claim 39, wherein the glycoconjugate is selected from the group consisting of an oligosaccharide, a glycoprotein, a glycolipid, a glycopeptide, and a steroid.

42. The method of claim 39, wherein the glycoconjugate comprises an oligosaccharide.

43. The method of claim 42, wherein the oligosaccharide comprises an α-Gal epitope.
